(19) United States
(12) Patent Application Publication
(10) Pub. No.: US 2024/0182991 A1
(43) Pub. Date: Jun. 6, 2024

(54) Colorimetric COVID-19 Rapid-Test and Methods of Using Same

(71) Applicant: YALE UNIVERSITY, New Haven, CT (US)

(72) Inventors: Chen Liu, New Haven, CT (US); Kien Pham, New Haven, CT (US); Jianhui Wang, Woodbridge, CT (US)

(21) Appl. No.: 18/556,077

(22) PCT Filed: Apr. 18, 2022
(86) PCT No.: PCT/US2022/025217

§ 371 Date: Oct. 18, 2023

Related U.S. Application Data
(60) Provisional application No. 62/176,512, filed on Feb. 20, 2015.

(51) Int. Cl. C12Q 1/70; C12Q 1/6806; C12Q 1/6844

(52) U.S. Cl. CPC C12Q 1/701; C12Q 1/6806; C12Q 1/6844; C12Q 2600/16

(57) ABSTRACT

The present disclosure provides a method of testing a subject for COVID-19, the method comprising the steps of: obtaining a saliva sample from the subject; diluting the saliva sample with a dilution buffer comprising a base; contacting the diluted saliva sample with one or more loop-mediated isothermal amplification (LAMP) primers that bind regions in the N gene of the SARS-CoV-2 virus to form a test mixture; and analyzing the color of the test mixture. The present disclosure also provides a COVID-19 rapid-test kit comprising one or more components for the collection of a saliva sample from a subject, one or more components for processing of the saliva sample, and one or more components used to interpret the results of the COVID-19 test.

### CROSS-REFERENCE TO RELATED APPLICATIONS

The present application claims priority under 35 U.S.C. § 119(e) to U.S. Provisional Patent Application No. 63/176,512, filed Apr. 19, 2021, which is hereby incorporated by reference in its entirety herein.

### BACKGROUND OF THE DISCLOSURE

The coronavirus disease 2019 (COVID-19) pandemic caused by the SARS-CoV-2 (severe acute respiratory syndrome coronavirus 2) coronavirus is a worldwide public health problem. The COVID-19 pandemic also has had an immense global impact through a variety of mediums—e.g., economic, psychological, public health, etc. In the United states, alone, there have been 29.8 million reported COVID-19 cases, and over 540,000 reported deaths. Further, while it is known that people with pre-existing conditions are at greater risk for COVID-19 related complications, all of the high-risk populations have yet to be identified. Moreover, studies show varying rates of asymptomatic spreaders across different populations, regularly projecting asymptomatic spreaders to account for 50% of all COVID-19 cases, underscoring the need for additional methods of containment for COVID-19. Additionally, among symptomatic populations, it has been reported that people are most contagious in the first 5 days after symptom onset, typically before presentation of severe symptoms, further compounding the level of complexity involved in early detection of COVID-19. This suggests that increased social distancing and mask-wearing efforts alone are not sufficient in curbing the spread of COVID-19, and that the early detection and quarantine of asymptomatic persons is needed. Furthermore, despite recent vaccination efforts in conjunction with stringent social distancing practices, large-scale, readily accessible rapid testing will be of paramount importance in slowing the spread of COVID-19 and returning to some resemblance of pre-pandemic life.

Rapid tests for detecting existing SARS-CoV-2 infections and assessing virus spread are critical. Unfortunately, there are no SARS-CoV-2 infection tests that are designed for a rapid on-site performance directly on saliva samples, without the need of RNA extraction or laboratory equipment. Such an on-site test would be particularly useful to detect infectious pathogen carriers, including apparent carriers who are asymptomatic. Current COVID-19 testing efforts rely upon sample collection of gross saliva and more invasive nasopharyngeal (NP) swabbing for the conventional methods of viral detection, such as reverse transcription quantitative polymerase chain reaction (RT-qPCR). The utilization of laboratory RT-qPCR requires an expensive and stationary collection of laboratory equipment, in addition to extended sample processing times. Typically, diagnostic laboratory testing utilizing RT-qPCR requires several processing steps, with unique reagents for each step, which often result in sample processing times that exceed several hours. Consequently, as testing demands increase, so to do turn-around-times for test results, which, when combined with reagent/supply shortages for the necessary equipment for this method of testing, leads to severely hindered throughput for clinical laboratory diagnostic testing. Therefore, development of rapid viral-genome amplification methods of testing has become the focus of testing efforts due to their increased throughput, their point-of-care (POC) capability, and potential for early detection of asymptomatic carriers of COVID-19.

Thus, there is a need in the art for methods that rapidly test a subject's saliva for SARS-CoV-2 infection. The present disclosure satisfies these unmet needs.

### SUMMARY OF THE DISCLOSURE

In one aspect, the invention provides a method of testing a subject for COVID-19. In certain embodiments, the method comprises obtaining a saliva sample from the subject. In certain embodiments, the method comprises diluting the saliva sample with a dilution buffer comprising a base. In certain embodiments, the method comprises contacting the diluted saliva sample with loop-mediated isothermal amplification (LAMP) primers that bind the N gene of the SARS-CoV-2 virus to form a test mixture under conditions that allow for reverse-transcription LAMP (RT-LAMP) of RNA material within the diluted saliva sample to take place. In certain embodiments, the LAMP primer comprises an inner primer. In certain embodiments, the reverse-transcribed RNA material comprises a first region which 5′ end is conjugated to the 3′ end of a second region which 5′ end is conjugated to the 3′ end of a DNA segment which is amplified by RT-LAMP. In certain embodiments, the 3′ end of inner primer comprises a first sequence that is complementary to the first region, and the first sequence is conjugated through its 5′ end to the 3′ end of a second sequence that is identical to the second region. In certain embodiments, a (A)ₙ polynucleotide, wherein n is 2, 3, 4, 5, 6, 7, 8, 9, or 10, is inserted between the first and second sequences within the inner primer. In certain embodiments, the method comprises analyzing the color of the test mixture.

In certain embodiments, the step of obtaining a saliva sample from the subject further comprises mixing the saliva sample with a lysis buffer.

In certain embodiments, the lysis buffer comprises at least one of a serine alkaline protease and a denaturing agent.

In certain embodiments, the serine alkaline protease is proteinase K and wherein the denaturing agent is guanidine hydrochloride.

In certain embodiments, the base is any alkaline or earth-alkaline hydroxide, such as but not limited to lithium hydroxide, sodium hydroxide, and/or potassium hydroxide, and/or ammonium hydroxide.

In certain embodiments, the LAMP primers comprises the primer: TCCCCTACTGCTGCCTGGAGGAAAACAGTCAAGCCTCTTCTCG (FIP-N1; SEQ ID NO:1). In certain embodiments, the LAMP primers comprises the primer: CTCCTGCTAGAATGGCTGGCAAAAATCTGTCAAGCAGCAGCAAAG (BIP-N1, SEQ ID NO:2). In certain embodiments, the LAMP primers comprises the primer: GCCAAAAGGCTTCTACGCA (F3-N1, SEQ ID NO:3). In certain embodiments, the LAMP primers comprises the primer: TTGCTCTCAAGCTGGTTCAA (B3-N1, SEQ ID NO:4). In certain embodiments, the LAMP primers comprises the primer: GCGACTACGTGATGAGGAA (LF-N1, SEQ ID NO:5). In certain embodiments, the LAMP primers comprises the primer: GGCGGTGATGCTGCTCTT (LB-N1, SEQ ID NO:6).

In certain embodiments, the LAMP primers comprises the primer: TGCGGCCAATGTTTGTAATCAGAAAACCAAGGAAATTTTGGGGAC (FIP-N2, SEQ ID NO:7). In certain embodiments, the LAMP primers comprises the primer: CGCATTGGCATGGAAGTCACAAAATTTGATGGCACCTGTGTAG (BIP-N2, SEQ ID NO:8). In certain embodiments, the LAMP primers comprises the primer:AACACAAGCTTTCGGCAG (F3-N2, SEQ ID NO:9). In certain embodiments, the LAMP primers comprises the primer: GAAATTTGGATCTTTGTCATCC (B3-N2, SEQ ID NO:10). In certain embodiments, the LAMP primers comprises the primer: TTCCTTGTCTGATTAGTTC (LF-N2, SEQ ID NO:11). In certain embodiments, the LAMP primers comprises the primer: ACCTTCGGGAACGTGGTT (LB-N2, SEQ ID NO:12).

In certain embodiments, the LAMP primers bind to two non-overlapping regions of the N gene of the SARS-CoV-2 virus.

In certain embodiments, the LAMP primers comprise each of SEQ ID NO: 1 to SEQ ID NO:12.

In certain embodiments, the step of contacting the diluted saliva sample with one or more LAMP primers to form a test mixture further comprises centrifuging the test mixture and heating the test mixture at about 65° C. for about 30 minutes.

In certain embodiments, the step of analyzing the color of the test mixture comprises comparing the color of the test mixture to a positive control and a negative control. In certain embodiments, when the test mixture has the same color as the negative control, the subject does not have COVID-19. In certain embodiments, the test mixture has the same color as the positive control, the subject has COVID-19. In certain embodiments, the test mixture has a color that is different than both the positive and negative controls, the result of the COVID-19 test is inconclusive.

In certain embodiments, when the COVID-19 test is inconclusive, the steps of obtaining a saliva sample from the subject, diluting the saliva sample with a dilution buffer comprising a base, and contacting the diluted saliva sample with one or more loop-mediated isothermal amplification (LAMP) primers that bind regions in the N gene of the SARS-CoV-2 virus to form a test mixture are repeated.

In certain embodiments, when the COVID-19 test is inconclusive, the step of contacting the diluted saliva sample with one or more LAMP primers to form a test mixture further comprises the steps of centrifuging the test mixture and heating the test mixture at about 65° C. for about 45 minutes.

In certain embodiments, when the COVID-19 test is inconclusive, the step of analyzing the color of the test mixture is repeated.

In another aspect, the invention provides a COVID-19 rapid-test kit comprising one or more components for the collection of a saliva sample from a subject, one or more components for processing of the saliva sample, and one or more components used to interpret the results of the COVID-19 test.

In certain embodiments, the one or more components for the collection of a saliva sample comprise a collection tube, lysis buffer comprising a serine alkaline protease, a denaturing agent, or a combination thereof, and an optional oral swab and compression tube.

In certain embodiments, the serine alkaline protease is proteinase K and the denaturing agent is guanidine hydrochloride.

In certain embodiments, the one or more components for processing of the saliva sample comprise a dilution buffer comprising a base, primer sets corresponding to:

(a) the set:
(FIP-N1; SEQ ID NO: 1)
TCCCCTACTGCTGCCTGGAGGAAAACAGTCAAGCCTCTTCTCG,
(BIP-N1, SEQ ID NO: 2)
CTCCTGCTAGAATGGCTGGCAAAAATCTGTCAAGCAGCAGCAAAG,
(F3-N1, SEQ ID NO: 3)
GCCAAAAGGCTTCTACGCA,
(B3-N1, SEQ ID NO: 4)
TTGCTCTCAAGCTGGTTCAA,
(LF-N1, SEQ ID NO: 5)
GCGACTACGTGATGAGGAA,
and
(LB-N1, SEQ ID NO: 6)
GGCGGTGATGCTGCTCTT;
and/or
(b) the set:
(FIP-N2, SEQ ID NO: 7)
TGCGGCCAATGTTTGTAATCAGAAAACCAAGGAAATTTTGGGGAC,
(BIP-N2, SEQ ID NO: 8)
CGCATTGGCATGGAAGTCACAAAATTTGATGGCACCTGTGTAG,
(F3-N2, SEQ ID NO: 9)
AACACAAGCTTTCGGCAG,
(B3-N2, SEQ ID NO: 10)
GAAATTTGGATCTTTGTCATCC,
(LF-N2, SEQ ID NO: 11)
TTCCTTGTCTGATTAGTTC,
and
(LB-N2, SEQ ID NO: 12)
ACCTTCGGGAACGTGGTT.

In certain embodiments, the kit comprises primers of each of SEQ ID NO:1 to SEQ ID NO: 12.

In certain embodiments, the one or more components for processing of the saliva sample further comprises one or more components for a positive and negative control. In certain embodiments, the one or more components for the negative control comprise DNA polymerase, reverse transcriptase, a pH indicator, dUTP, and Uracil-DNA Glycosylase (UDG). In certain embodiments, the one or more components for the positive control comprise DNA polymerase, reverse transcriptase, a pH indicator, dUTP, UDG, and RNase P gene primers selected from the group consisting of:

(FIP-R, SEQ ID NO: 13)
GTGTGACCCTGAAGACTCGGAAAAAGCCACTGACTCGGATC,
(BIP-R, SEQ ID NO: 14)
CCTCCGTGATATGGCTCTTCGAAAATTTCTTACATGGCTCTGGTC,
(F3-R, SEQ ID NO: 15)
TTGATGAGCTGGAGCCA,
(B3-R, SEQ ID NO: 16)
CACCCTCAATGCAGAGTC,
(LF-R, SEQ ID NO: 17)
ATGTGGATGGCTGAGTTGTT,
and
(LB-R, SEQ ID NO: 18)
CATGCTGAGTACTGGACCTC.

In certain embodiments, the negative control and the positive control both further comprise a portion of the saliva sample obtained from the subject in the dilution buffer.

In certain embodiments, the one or more components used to interpret the results of the COVID-19 test comprises a card showing the expected color of a positive, negative, and inconclusive COVID-19 test result.

### BRIEF DESCRIPTION OF THE DRAWINGS

The following detailed description of selected embodiments of the invention will be better understood when read in conjunction with the appended drawings. For the purpose of illustrating the invention, selected embodiments are shown in the drawings. It should be understood, however, that the invention is not limited to the precise arrangements and instrumentalities of the embodiments shown in the drawings.

FIG. 1 depicts an illustrative card for reporting the results of the SaliVISION™ test.

FIGS. 2A-2B depict the neutralization of Acidic Saliva Using NaOH Solution. FIG. 2A: Acidic (low pH) and normal (neutral pH) saliva samples were diluted with different concentrations of NaOH solution. The picture was taken immediately after the dilution. The test was performed with 3 independent samples for each group. FIG. 2B: Acidic and normal saliva samples were spiked with 20 copies/μl of synthetic SARS-CoV-2 RNA and then diluted in different concentrations of NaOH solution. The spiked samples were then processed with SaliVISION test at 65° C. for 30 minutes. The final copy number of synthetic viral RNA was 50 copies per reaction. The red box indicates the optimal concentration of NaOH solution (Dilution Buffer) that meets requirement for both specificity and sensitivity. The images were taken with a phone camera. The tests were repeated 3 times, independently.

FIGS. 3A-3D depict the SARS-CoV-2 Detection Limit of the SaliVISION™ Test Using Saliva Samples. Different concentrations of synthetic SARS-CoV-2 RNA was spiked in Dilution Buffer. FIG. 3A: Positive saliva samples subjected to RT-LAMP analysis with SaliVISION™ test. The reactions were incubated at 65° C. for 30 minutes. The color change in each sample was captured immediately after the reactions were terminated, using a phone camera. FIG. 3B: A summary of positive reactions of 10 independent replicates of viral RNA directly added to Dilution Buffer. FIG. 3C: Negative saliva samples subjected to RT-LAMP analysis with SaliVISION™ test. The reactions were incubated at 65° C. for 30 minutes. The color change in each sample was captured immediately after the reactions were terminated, using a phone camera. FIG. 3D: A summary of 20 negative saliva sampled spiked with viral RNA directly added to Dilution Buffer. The provided number in FIGS. 3A-3D represent the actual copy number of viral RNA in each SaliVISION™ reaction.

FIGS. 4A-4C depict the clinical validation of SaliVISION™, tested side-by-side with SalivaDirect™, using saliva samples. Saliva samples being collected for clinical diagnostic testing were subjected to SARS-CoV-2 detection using SaliVISION™ (RT-LAMP) and SalivaDirect™ (RT-qPCR) methods. In both methods, saliva samples were treated with the lysis buffer and heat-inactivated before being processed. The results of RT-LAMP are compared to relative Ct value determined by quantitative RT-PCR SalivaDirect™ test. FIG. 4A: The distribution of negative (pink) and positive (yellow) samples detected by the SaliVISION™ test according to their corresponding Ct value detected by the SalivaDirect test (Y axis). The dotted line indicates the cut-off value for SalivaDirect™ test, in which the samples with Ct value>40 is considered negative and that with Ct value≤40 is considered positive. FIG. 4B: Specificity (left) and sensitivity (right) of the SaliVISION™ RT-LAMP assay, derived from data in FIGS. 3A and 3C, are shown. For sensitivity, the RT-qPCR positive samples were stratified into four groups, based on the Ct value (x axis). The red lines indicate the value of these proportions. The floating boxes indicate the corresponding 95% confidence intervals computed by Wald's method. FIG. 4C: A summary of correlative comparison between the results of SaliVISION™ and SalivaDirect™ on the same sample set.

FIGS. 5A-5C depict clinical validation of SaliVISION™, tested side-by-side with TaqPath™, using saliva samples. Saliva samples being collected for clinical diagnostic testing were subjected to SARS-CoV-2 detection using SaliVISION™ (RT-LAMP) and ThermoFisher TaqPath™ (RT-qPCR) methods. In both methods, saliva samples were treated with the lysis buffer and heat-inactivated before being processed. The results of RT-LAMP are compared to relative Ct value determined by quantitative TaqPath™ RT-qPCR test. FIG. 5A: The distribution of negative (pink) and positive (yellow) samples detected by the SaliVISION™ test according to their corresponding Ct value detected by the TaqPath™ test (Y axis). The dotted line indicates the cut-off value for SalivaDirect™ test, in which the samples with Ct value>40 is considered negative and that with Ct value≤40 is considered positive. FIG. 5B: Specificity (left) and sensitivity (right) of the SaliVISION™ RT-LAMP assay, derived from data in FIGS. 5A and 5C, are shown. For sensitivity, the RT-qPCR positive samples were stratified into four groups, based on the Ct value (x axis). The red lines indicate the value of these proportions. The floating boxes indicate the corresponding 95% confidence intervals computed by Wald's method. FIG. 5C: A summary of correlative comparison between the results of SaliVISION™ and TaqPath™ on the same sample set.

FIGS. 6A-6C depict the clinical validation of SaliVISION™, tested side-by-side with TaqPath™, using saliva and nasopharyngeal Swab Samples, respectively. Pairs of saliva and nasopharyngeal (NP) swab samples being collected from the study participants were subjected to SARS-CoV-2 detection using SaliVISION™ (RT-LAMP) and ThermoFisher TaqPath™ (RT-qPCR) methods, respectively. For SaliVISION™ test, saliva samples were treated with the lysis buffer and heat-inactivated before being processed as described previously. This test was performed on-site immediately after the collection. The NP swab samples were processed according to standard protocol as described previously in the following day. The results of RT-LAMP are compared to relative Ct value determined by quantitative RT-PCR TaqPath™ test. FIG. 6A: The distribution of negative (pink) and positive (yellow) samples detected by the SaliVISION™ test according to their corresponding Ct value detected by the TaqPath™ test (Y axis). The dotted line indicates the cut-off value for TaqPath™ test, in which the samples with Ct value>40 is considered negative and that with Ct value≤40 is considered positive. FIG. 6B: Specificity (left) and sensitivity (right) of the SaliVISION™ RT-LAMP assay, derived from data in FIGS. 6A and 6C, are shown. For sensitivity, the RT-qPCR positive samples were stratified into four groups, based on the Ct value (x axis). The red lines indicate the value of these proportions. The floating boxes indicate the corresponding 95% confidence intervals computed by Wald's method. FIG. 6C: A summary of correlative comparison between the results of SaliVISION™ and TaqPath™ on the same sample set.

FIGS. 7A-7B are tables showing an in silico cross-reactivity of analysis of the SaliVISION™ multiplex primer set.

FIG. 8 is a table of the commercially available pathogen panels for cross-reactivity wet testing with the SaliVISION™ assay.

FIGS. 9A-9B are tables showing the wet testing result of the cross-reactivity of SaliVISION™.

FIG. 10 depicts the SaliVISION™ COVID-19 Screening and Diagnostic Approach. 1) The saliva sample self-collected with passive drooling or with MicroSAL™ Saliva Collection Kit is placed in a Collection Tube pre-loaded with Lysis Buffer. 2) The collected saliva sample in the Collection Tube is lysed and heat-inactivated at 95° C. in 10 minutes. 3) A fixed volume of inactivated saliva is processed through a serial dilution step in a microtube test strip pre-loaded with Dilution Buffer (#0), negative control (#1), SARS-CoV-2 test with multiplex primers targeting the viral N gene (#2), and internal control with human RNP gene (#3). The RT-PCR reaction will be processed at 65° C. for 30 minutes. 4) The result is read and interpreted with the cooled reaction mixes after a 30-minute incubation with a REPORT CARD.

FIG. 11 depicts a table illustrating in silico Inclusivity Analysis with SaliVISION Multiplex Primer Set Targeting N gene of SARS-CoV-2 virus.

### DETAILED DESCRIPTION

In one aspect, the present disclosure relates to a method of testing a subject for SARS-CoV-2 infection. In certain embodiments, the method comprises obtaining a saliva sample from the subject. In certain embodiments, the method comprises diluting the saliva sample with a dilution buffer comprising a base. In certain embodiments, the method comprises contacting the diluted saliva sample with loop-mediated isothermal amplification (LAMP) primers that bind a region in the N gene of the SARS-CoV-2 virus to form a test mixture under conditions that allow for reverse-transcription LAMP (RT-LAMP) of RNA material within the diluted saliva sample to take place. In certain embodiments, the method comprises analyzing the color of the test mixture.

In some embodiments, the step of obtaining a saliva sample from the subject further comprises the step of mixing the saliva sample with a lysis buffer. In certain embodiments, the lysis buffer comprises a protease and/or a denaturing agent.

The disclosure contemplates that the reverse-transcribed RNA material to be amplified comprises a first region which 5′ end is conjugated to the 3′ end of a second region which 5′ end is conjugated to the 3′ end of the target DNA sequence which is amplified by RT-LAMP. According to the LAMP technology, each inner primer comprises at the 3′ end a first sequence that is complementary to the first region described above, and the first sequence is conjugated through its 5′ end to the 3′ end of a second sequence that is identical to the second region described above. According to the present disclosure, the inner primer are designed to comprise a polyA segment inserted between the first and second sequences. Although not wishing to be limited by theory, it is believed that the length of the poly A segment should be optimized such that the segment provides enough space to increase the specificity but is not too long to reduce its sensitivity during the annealing process. In some embodiment, the polyA segment contains 2, 3, 4, 5, 6, 7, 8, 9, or 10 A nucleotides. In certain embodiments, the polyA segment is an AAAA polynucleotide. In certain embodiments, this polyA segment more efficiently eliminates or minimizes any potential contaminating amplicons because its use enhances the number of uracil residues incorporated into the amplified products. Therefore, if there is any contaminating dU-containing product from previous reactions, it will be recognized and removed by Uracil-DNA Glycosylase (UDG) in the SaliVISION™ assay disclosed herein.

In certain embodiments, the LAMP primers comprise the set of primers corresponding to SEQ ID NO:1 to SEQ ID NO:6. In certain embodiments, the LAMP primers comprise the set of primers corresponding to SEQ ID NO:7 to SEQ ID NO: 12. In some embodiments, the LAMP primers comprise a primer of each of SEQ ID NO: 1 to SEQ ID NO:12. In some embodiments, the lowest concentration of SARS-CoV-2 RNA that can be detected by the method disclosed herein is about 2.5 copies of SARS-CoV-2 RNA/μL of sample.

In another aspect, the present disclosure relates to a COVID-19 rapid-test kit. In some embodiments, the kit comprises components used for saliva collection, components used for processing of the saliva sample, and components used to interpret the results of the COVID-19 test. In some embodiments, the kit further comprises instructions for saliva collection, processing the saliva sample, and interpreting the test results. In some embodiments, the lowest concentration of SARS-CoV-2 RNA that can be detected by the kit disclosed herein is about 2.5 copies of SARS-CoV-2 RNA/μL of sample.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element. “About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Amplification” refers to any means by which a polynucleotide sequence is copied and thus expanded into a larger number of polynucleotide sequences, e.g., by reverse transcription, polymerase chain reaction or ligase chain reaction, among others.

The term “assessing” includes any form of measurement, and includes determining if an element is present or not. The terms “determining,” “measuring,” “evaluating,” “assessing,” and “assaying” are used interchangeably and include quantitative and qualitative determinations. Assessing may be relative or absolute. “Assessing the presence of” includes determining the amount of something present, and/or determining whether it is present or absent.

“Detect” refers to identifying the presence, absence, level, or concentration of an agent.

In the context of the present invention, the following abbreviations for the commonly occurring nucleic acid bases are used. “A” refers to adenosine, “C” refers to cytosine, “G” refers to guanosine, and “T” refers to thymidine.

As used herein, the term “LAMP” or “Loop-mediated isothermal amplification” refers to a single-tube technique for the amplification of DNA. Reverse transcription loop-mediated isothermal amplification (RT-LAMP) combines LAMP with a reverse transcription step to allow the detection of RNA. LAMP is an isothermal nucleic acid amplification technique: it is carried out at a constant temperature, and does not require a thermal cycler. In LAMP, the target sequence is amplified at a constant temperature of 60-65° C. using either two or three sets of primers and a polymerase with high strand displacement activity in addition to a replication activity. Typically, four different primers are used to amplify six distinct regions on the target gene, which increases specificity. An additional pair of “loop primers” can further accelerate the reaction. See, for example, Notomi, et al., 2000, Nucleic Acids Res. 28 (12): 63e-63; and U.S. Pat. No. 6,41,0278, the contents of each of which are incorporated herein in their entireties by reference.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Methods of Testing a Subject for COVID-19

In one aspect, the present disclosure relates to a method of testing a subject for SARS-CoV-2 infection. In certain embodiments, the method comprises the steps of: obtaining a saliva sample from the subject, diluting the saliva sample with a dilution buffer comprising a base, contacting the diluted saliva sample with loop-mediated isothermal amplification (LAMP) primers that bind two non-overlapping regions in the N gene of the SARS-CoV-2 virus to form a test mixture, and analyzing the color of the test mixture.

The subject can be any subject known to a person of skill in the art. In certain embodiments, the subject has one or more symptoms that can be indicative of a SARS-CoV-2 infection. In other embodiments, the subject is asymptomatic. In certain embodiments, the subject is a human subject. The saliva sample can be obtained from the subject using any method known to a person of skill in the art. In certain embodiments, the subject allows saliva to pool in his or her mouth and the saliva sample is obtained with an oral swab, such as a cotton swab. In other embodiments, the subject allows saliva to pool in his or her mouth and then the subject spits the saliva sample into a collection tube.

In some embodiments, the step of obtaining a saliva sample from the subject further comprises the step of mixing the saliva sample with a lysis buffer. In certain embodiments, the lysis buffer comprises a protease, a denaturing agent, or a combination thereof. In certain embodiments, the protease is a serine alkaline protease. In some embodiments, the serine alkaline protease has been FDA approved to treat saliva. In certain embodiments, the serine alkaline protease is proteinase K (PK). In other embodiments, the serine alkaline protease is Qiagen Protease. In certain embodiments, the denaturing agent can be any denaturing agent known to a person of skill in the art to homogenize cells and denature RNases. In some embodiments, the denaturing agent is guanidine hydrochloride (GHCl). In other embodiments, the denaturing agent is guanidine thiocyanate (GuSCN). In certain embodiments, the lysis buffer comprises PK and GHCl. Although not wishing to be limited by theory, it is believed that the lysis buffer promotes cell dissociation and RNA stabilization. Although not wishing to be limited by theory, it is believed that, if the saliva sample is not mixed with a denaturing agent, there will be inefficient cell dissociation (that requires a longer reaction time and/or higher concentrations of protease) and a reduction in RNA integrity during the processing of the saliva sample. In certain embodiments wherein the saliva sample is obtained by having the subject spit into a collection tube, the subject spits the saliva sample into a tube containing the lysis buffer, therefore allowing the saliva sample to mix with the lysis buffer. In other embodiments wherein the saliva sample is obtained using an oral swab, the saliva collected on the oral swab is transferred into a collection tube containing the lysis buffer. In some embodiments, the collection tube comprising the lysis buffer is heated before mixing with the saliva sample. In certain embodiments, the collection tube comprising the lysis buffer is heated at about 95° C. with shaking at about 1000 rpm before mixing with the saliva sample.

The step of diluting the saliva sample with a dilution buffer comprising a base can use any base known to a person of skill in the art. Although not wishing to be limited by theory, it is believed that the base functions to neutralize the natural acidity of the saliva sample, stabilizes the pH before reaction with the LAMP primers, and decreases the chances of having false positive or invalid COVID-19 test results. The dilution buffer comprising a base can comprise a low concentration of any strong base known to a person of skill in the art. In certain embodiments, the base is sodium hydroxide. In certain embodiments, the dilution buffer comprises between about 500 μM and 2.5 mM, about 500 μM and 2 mM, about 750 μM and 2 mM, or about 750 μM and 1.5 mM of a strong base. In some embodiments, the dilution buffer comprises about 1 mM of a strong base. In some embodiments, the dilution buffer comprises about 1 mM of sodium hydroxide.

The step of contacting the diluted saliva sample with loop-mediated isothermal amplification (LAMP) primers that bind regions in the N gene of the SARS-CoV-2 virus can use any LAMP primers known to a person of skill in the art to bind regions in the N gene of the SARS-CoV-2 virus.

In certain embodiments, the LAMP primers comprise one set of primers corresponding to:

(FIP-N1; SEQ ID NO: 1)
TCCCCTACTGCTGCCTGGAGGAAAACAGTCAAGCCTCTTCTCG,
(BIP-N1, SEQ ID NO: 2)
CTCCTGCTAGAATGGCTGGCAAAAATCTGTCAAGCAGCAGCAAAG,
(F3-N1, SEQ ID NO: 3)
GCCAAAAGGCTTCTACGCA,
(B3-N1, SEQ ID NO: 4)
TTGCTCTCAAGCTGGTTCAA,
(LF-N1, SEQ ID NO: 5)
GCGACTACGTGATGAGGAA,
and
(LB-N1, SEQ ID NO: 6)
GGCGGTGATGCTGCTCTT.

In certain embodiments, the LAMP primers of SEQ ID NO: 1 to SEQ ID NO: 6 bind to the N.1 region of the SARS-CoV-2 N gene.

In certain embodiments, the LAMP primers comprise one set of primers corresponding to:

(FIP-N2, SEQ ID NO: 7)
TGCGGCCAATGTTTGTAATCAGAAAACCAAGGAAATTTTGGGGAC,
(BIP-N2, SEQ ID NO: 8)
CGCATTGGCATGGAAGTCACAAAATTTGATGGCACCTGTGTAG,
(F3-N2, SEQ ID NO: 9)
AACACAAGCTTTCGGCAG,
(B3-N2, SEQ ID NO: 10)
GAAATTTGGATCTTTGTCATCC,
(LF-N2, SEQ ID NO: 11)
TTCCTTGTCTGATTAGTTC,
and
(LB-N2, SEQ ID NO: 12)
ACCTTCGGGAACGTGGTT.

In certain embodiments, the LAMP primers of SEQ ID NO: 7 to SEQ ID NO: 12 bind to the N.2 region of the SARS-CoV-2 N gene.

In certain embodiments, the LAMP primers comprise each of SEQ ID NO: 1 to SEQ ID NO: 12. Although not wishing to be limited by theory, it is believed that the use of each of SEQ ID: 1 to SEQ ID NO: 12 produces an intensified signal and therefore increases the sensitivity of the COVID-19 test. In some embodiments, the use of each of SEQ ID NO: 1 to SEQ ID NO:12 increases the specificity of the COVID-19 test such that there is no cross-reactivity to common respiratory or other viral pathogens. In some embodiments, DNA polymerase, reverse transcriptase, a pH indicator, dUTP, UDGs, or a combination thereof, is added to the test mixture. In certain embodiments, each of DNA polymerase, reverse transcriptase, a pH indicator, dUTP, and UDGs is added to the test mixture. In some embodiments, the pH indicator is phenol red. In some embodiments, WarmStart® LAMP 2× Master Mix is added to the test mixture. WarmStart® LAMP 2× Master Mix contains a blend of BST 2.0 WarmStart DNA polymerase and WarmStart RTx Reverse Transcriptase for the amplification, pH indicator phenol red for result visualization, and dUTP and UDGs for prevention of carryover contamination. In certain embodiments, after contacting the diluted saliva sample with the LAMP primers to form a test mixture, the test mixture is centrifuged for about five seconds and then heated. In some embodiments, the test mixture is heated at about 65° C. for about 30 minutes.

The step of analyzing the color of the test mixture can use any method known to a person of skill in the art. In certain embodiments, the color of the test mixture is analyzed visually by a trained staff member who is administering the test. In certain embodiments, the color of the test mixture is different for a subject who has a positive COVID-19 test result compared to a subject who has a negative COVID-19 test result. In certain embodiments, a pink/red color indicates that the subject does not have COVID-19 (i.e. a negative COVID-19 test result). In other embodiments, a yellow color indicates that the subject has COVID-19 (i.e. a positive COVID-19 test result). In other embodiments, an orange color indicates that the test is inconclusive. In some embodiments, when the result of the COVID-19 test is inconclusive, the steps of the above method are repeated and, after contacting the diluted saliva sample with the LAMP primers to form a second test mixture, the second test mixture is centrifuged for about five seconds and then heated at about 65° C. for about 45 minutes. The color of the second test mixture is then analyzed as described above for the test mixture. If the result of the COVID-19 test remains inconclusive, the subject's COVID-19 test result is reported is reported as inconclusive. Therefore, in some embodiments, the method further comprises referring a subject for whom the above method has led to two inconclusive COVID-19 test results to a COVID-19 testing location which uses a different testing method than the one disclosed herein.

In some embodiments, the step of analyzing the color of the test mixture comprises comparing the color of the mixture to a positive control and a negative control. In some embodiments, the negative control comprises a portion of the saliva sample obtained from the subject in the dilution buffer described elsewhere herein and WarmStart® LAMP 2× Master Mix. In embodiments wherein the above method comprises the step of mixing the saliva sample with a lysis buffer, the negative control comprises the lysis buffer described elsewhere herein. In some embodiments, the positive control comprises a portion of the saliva sample obtained from the subject in the dilution buffer described elsewhere herein, WarmStart® LAMP 2× Master Mix, and one or more RNase P gene primers. In embodiments wherein the above method comprises the step of mixing the saliva sample with a lysis buffer, the positive control comprises the lysis buffer described elsewhere herein. In some embodiments, the RNase P gene primers comprise one or more of the following primers:

(FIP-R, SEQ ID NO: 13)
GTGTGACCCTGAAGACTCGGAAAAAGCCACTGACTCGGATC,
(BIP-R, SEQ ID NO: 14)
CCTCCGTGATATGGCTCTTCGAAAATTTCTTACATGGCTCTGGTC,
(F3-R, SEQ ID NO: 15)
TTGATGAGCTGGAGCCA,
(B3-R, SEQ ID NO: 16)
CACCCTCAATGCAGAGTC,
(LF-R, SEQ ID NO: 17)
ATGTGGATGGCTGAGTTGTT,
and
(LB-R, SEQ ID NO: 18)
CATGCTGAGTACTGGACCTC.

In certain embodiments, the positive control comprises each of SEQ ID NO: 13 to SEQ ID NO:18.

In some embodiments, the negative control and positive control are each centrifuged and heated as described above for the test mixture. In certain embodiments, the negative control should have a pink/red color and the positive control should have a yellow color. In certain embodiments wherein either the negative control is not pink/red or the positive control is not yellow, the COVID-19 test is invalid and above method does not comprise the step of analyzing the color of the test mixture.

In some embodiments, the method can detect a concentration of SARS-CoV-2 of at least 2.5 copies/μL in the saliva sample obtained from the subject.

### COVID-19 Testing Kits

In another aspect, the present disclosure relates to a COVID-19 rapid-test kit. In certain embodiments, the COVID-19 rapid-test kit comprises components used for saliva collection, components used for processing of the saliva sample, and components used to interpret the results of the COVID-19 test. In some embodiments, the kit further comprises instructions for saliva collection, processing the saliva sample, and interpreting the test results.

### Components for Saliva Collection

In certain embodiments, the kit comprises a collection tube. In some embodiments, the collection tube is prefilled with lysis buffer described elsewhere herein. In other embodiments, the kit comprises the protease and/or denaturing agent to be used in the lysis buffer such that the lysis buffer can be prepared at the testing site. In some embodiments, the kit comprises an oral swab which is used to collect the saliva sample from a subject's mouth. In some embodiments wherein the kit comprises an oral swab, the kit further comprises a compression tube which fits into the top of the collection tube and is in communication with the collection tube. In some embodiments, the compression tube comprises a plunger. In certain embodiments, the oral swab is placed into the compression tube, the plunger is depressed, and the subject's saliva sample from the oral swab is transferred from the oral swab through the compression tube and into the collection tube. In some embodiments, the components for saliva collection further comprise instructions for collecting a saliva sample from a subject either using the oral swab or by having the subject spit into the collection tube.

### Components for Saliva Sample Processing

In certain embodiments, the kit comprises the dilution buffer described elsewhere herein or the base to be used in the dilution buffer such that the dilution buffer can be prepared at the testing site. In some embodiments, the kit comprises components to detect SARS-CoV-2 in the subject's saliva sample by LAMP technology. Therefore, in some embodiments, the kit comprises DNA polymerase, reverse transcriptase, a pH indicator, dUTP, and UDGs. In some embodiments, the kit comprises phenol red pH indicator. In some embodiments, the kit comprises WarmStart® LAMP 2× Master Mix, which comprises DNA polymerase, reverse transcriptase, phenol red pH indicator, dUTP, and UDGs. In certain embodiments, the kit comprises one or more forward primers that bind regions in the N gene of the SARS-CoV-2 virus, and one or more reverse primers that bind regions in the N gene of the SARS-CoV-2 virus. In some embodiments, the one or more forward primers are selected from SEQ ID NO: 1 to SEQ ID NO:6 described elsewhere herein. In some embodiments, the one or more reverse primers are selected from SEQ ID NO:7 to SEQ ID NO: 12 described elsewhere herein. In some embodiments, the kit comprises primers of each of SEQ ID NO:1 to SEQ ID NO: 12. In some embodiments, the kit further comprises components for a positive and negative control. Therefore, in some embodiments, the kit comprises DNA polymerase, reverse transcriptase, a pH indicator, dUTP, and UDGs to be used in both the positive and negative controls as well as one or more RNase P gene primers to be used in the positive control. In other embodiments, the kit comprises WarmStart® LAMP 2× Master Mix to be used in both the positive and negative controls as well as one or more RNase P gene primers to be used in the positive control. In some embodiments, the RNase P gene primers are selected from SEQ ID NO: 13 to SEQ ID NO:18 described elsewhere herein. In certain embodiments, the kit comprises primers of each of SEQ ID NO: 13 to SEQ ID NO:18. In some embodiments, the components for saliva sample processing further comprise instructions for preparing the positive and negative controls as well as instructions detailing how to test the subject's saliva sample for SARS-CoV-2 infection.

### Components for Interpreting the Test Results

In certain embodiments, the kit comprises a card for reporting the results of the subject's COVID-19 test. In some embodiments, the card comprises a section showing the expected color of a positive, negative, and inconclusive COVID-19 test result. In some embodiments, the card comprises a section showing the expected color of the negative and positive controls. In some embodiments, the components for interpreting the test results further comprise instructions for how to visually interpret the color of the subject's COVID-19 test.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1: SaliVISION: An On-Site Rapid Test for Human Pathogens With a High Efficiency in Cost, Time, Sensitivity, and Specificity

### Materials and Methods

SaliVISION™ is a Reverse Transcription Loop-mediated Isothermal Amplification (RT-LAMP) test intended for the qualitative detection of SARS-CoV-2 RNA in saliva samples. The SARS-CoV-2 RNA is generally detectable in respiratory specimens during the acute phase of infection, including saliva specimens. SaliVISION™ is intended for use of detection of SARS-CoV-2 RNA, by qualified and trained clinical laboratory personnel specifically instructed and trained in the techniques of RT-qPCR and in vitro diagnostic procedures. The assay is only for use under the Food and Drug Administration's Emergency Use Authorization.

Positive results do not rule out bacterial infection or co-infection with other viruses. Laboratories are required to report all positive results to the appropriate public health authorities.

Negative results do not preclude SARS-CoV-2 infection and should not be used as the sole basis for patient management decisions. Negative results must be combined with clinical observations, patient history, and epidemiological information. Negative results for SARS-CoV-2 RNA from saliva should be confirmed by testing of an alternative specimen type if clinically indicated.

TABLE 1
Equipment, reagents and materials

| | |
|---|---|
| Catalog # | Manufacturer |

EQUIPMENT AND CONSUMABLES

| | | |
|---|---|---|
| Digital dry heat block or | N/A | Any brand |
| water bath | | |
| 1.5- or 2.0-mL Eppendorf | 13-698-791/2 | ThermoFisher |
| DNA LoBind Tubes or Screw | | |
| Cap Tube | | |
| MicroSAL ™ Saliva | Oasis | |
| Collection Kits | Diagnostics | |
| TempAssure PCR flex-free | 1402-2300 | USA |
| 8-tube strips with attached | | Scientific |
| optical caps | | |
| Cool blocks for 1.5 mL and | N/A | Any brand |
| 0.2 mL microcentrifuge tubes | | |
| Capper/Decapper for 0.2 | N/A | Any brand |
| mL tubes | | |
| Vacuum sealer | N/A | Any brand |
| Vacuum sealer bags, | N/A | |
| 4″ × 6″ | | |

REAGENTS

| | | |
|---|---|---|
| Proteinase K (Tritirachium | PR-V3021 | Promega |
| album) | | |
| Proteinase K (Tritirachium | AB00925 | AmericanBio |
| album) | | |
| WarmStart ® Colorimetric | M1804L | NEB |
| LAMP 2x Master Mix with UDG | | |
| Nuclease-Free Water (Not | AM9937 | Invitrogen |
| DEPC Treated) | | |
| 100 μM desalted, custom | N/A | IDT |
| synthesized oligos | | |
| Sodium hydroxide, anhdryous, | 795429-500G | Sigma |
| pellets, ≥97% or equivalent | | |
| PBS, without Ca⁺⁺, | 21-040-CV | Corning |
| Mg⁺⁺ | | |
| Guanidine Hydrochloride, | 50937-100ML | Sigma |
| 8M in H₂O | | |

TABLE 2
Lysis buffer (LB)

| | | | Final |
|---|---|---|---|
| Component | | | Concentration |
| Proteinase K | 100 | mg | 10 mg/mL |
| 8M Guanidine HCl in | 0.5 | mL | 0.4M |
| H₂O, pH = 8 | | | |
| PBS | 5 | mL | |
| Total | 10 | mL | |

Prepare 10 mL of 4M Guanidine HCl in PBS → add 100 mg
of Proteinase K filter with 0.2 μm filter → aliquot
100 μl to each collection tube → stored at −20° C.

TABLE 3
10X multi-plex primer panels
N GENE

| | 100 μM | |
|---|---|---|
| Primer | stock (μl) | Oligo Sequence |
| FIP-N1 | 160 | TCCCCTACTGCTGCCTGGAGGAAAACAGTCAAGCCTCTTC |
| | | TCG (SEQ ID NO: 1) |
| BIP-N1 | 160 | TCTCCTGCTAGAATGGCTGGCAAAAATCTGTCAAGCAGCA |
| | | GCAAAG (SEQ ID NO: 2) |
| F3-N1 | 20 | GCCAAAAGGCTTCTACGCA (SEQ ID NO: 3) |
| B3-N1 | 20 | TTGCTCTCAAGCTGGTTCAA (SEQ ID NO: 4) |
| LF-N1 | 40 | GCGACTACGTGATGAGGAA (SEQ ID NO: 5) |
| LB-N1 | 40 | GGCGGTGATGCTGCTCTT (SEQ ID NO: 6) |
| FIP-N2 | 160 | TGCGGCCAATGTTTGTAATCAGAAAACCAAGGAAATTT |
| | | TGGGGAC (SEQ ID NO: 7) |
| BIP-N2 | 160 | CGCATTGGCATGGAAGTCACAAAATTTGATGGCACCTG |
| | | TGTAG (SEQ ID NO: 8) |
| F3-N2 | 20 | AACACAAGCTTTCGGCAG (SEQ ID NO: 9) |
| B3-N2 | 20 | GAAATTTGGATCTTTGTCATCC (SEQ ID NO: 10) |
| FL-N2 | 80 | TTCCTTGTCTGATTAGTTC (SEQ ID NO: 11) |
| LB-N2 | 80 | ACCTTCGGGAACGTGGTT (SEQ ID NO: 12) |
| H₂O | 40 | |
| Total | 1000 | |

RNase P GENE

| | 100 μM | |
|---|---|---|
| Primer | stock (μl) | Oligo Sequence |
| FIP-R | 160 | GTGTGACCCTGAAGACTCGGAAAAAGCCACTGACTCGG |
| | | ATC (SEQ ID NO: 13) |
| BIP-R | 160 | CCTCCGTGATATGGCTCTTCGAAAATTTCTTACATGGCT |
| | | CTGGTC (SEQ ID NO: 14) |
| F3-R | 20 | TTGATGAGCTGGAGCCA (SEQ ID NO: 15) |
| B3-R | 20 | CACCCTCAATGCAGAGTC (SEQ ID NO: 16) |
| LF-R | 40 | ATGTGGATGGCTGAGTTGTT (SEQ ID NO: 17) |
| LB-R | 40 | CATGCTGAGTACTGGACCTC (SEQ ID NO: 18) |
| H₂O | 560 | |
| Total | 1000 | |

TABLE 4
RT-LAMP reaction mix setup (per sample)

| | Dilution | Negative | | Positive |
|---|---|---|---|---|
| | Buffer | Control | Test | Control |
| Component | (#0) | (#1) | (#2) | (#3) |
| WarmStart ® LAMP | | 60 | | |
| 2X Master Mix | | | | |
| 1 mM NaOH | 60 | | | |
| PK-Treated Saliva | 30 | | | |
| Sample | | | | |
| 10X N-GENE Primer | | | 4 | |
| Mix | | | | |
| 10X RNase P-GENE | | | | 4 |
| Primer Mix | | | | |
| 1M Guanidine HCl | | | 1 | |
| Nuclease-Free Water | | | 5 | 6 |

Prepare a master mix for #2 and #3 → Aliquot
reagents accordingly to 0.2 mL PCR test strips in a
clean PCR hood, with minimal exposure to the air
flow and potential contaminations → air vacuum
and seal in appropriate storage bags → stored
at −20° C. until use within 1-2 days.
Pay extra attention to exposing the WarmStart ® Master
Mix to the air or any source that may impact the pH.
On-site storage: The aliquoted reaction mix can be
stored on dry ice ONLY when it is vacuum sealed.

### Clinical Sample Collection and Handling

The biospecimens in this study were obtained from both retrospective and prospective collections. For retrospective collection, this study used leftover nasopharyngeal swab and saliva samples that were previously collected for the clinical diagnosis of COVID-19. For prospective collection (on-site study), saliva samples were donated by symptomatic individuals who came to a testing site for screening. All participating subjects gave verbal consent with a study's information sheet and a waiver of a signed consent. Nasopharyngeal swab (NP) samples were collected and transported in 3 mL of viral transported medium (VTM), according to CDC and FDA guideline. This collection is a part of routine clinical testing offered at the selected testing sites. For saliva collection, the specimens were obtained from either passive drool or with a Micro·SAL Saliva Collection Kit (Oasis Diagnostics, Vancouver, WA), according to the manufacturer's instruction. Saliva samples collected from volunteers at the testing sites were processed on-site within 15 minutes, while those collected for the COVID-19 clinical diagnosis laboratory were processed 1-2 days after collection.

### Sample Lysis With Lysis Buffer

Upon collection, approximately 500 μl of saliva was aliquoted into a 1.5 mL Eppendorf microcentrifuge tube pre-loaded with 100 μL of lysis buffer containing 10 mg/mL of Proteinase K (AmericanBio, Canton, MA)) and 2M Guanidine hydrochloride (Sigma Aldrich, St. Louis, MO) diluted in sterile PBS. Once collected, saliva samples were lysed for 1 minute at 1500 rpm. The lysed saliva samples were subsequently heated inactivated for 5 minutes at 95° C. After the samples were cooled down, the treated samples were subjected to SaliVISION™ RT-LAMP and Thermo Fisher TaqPath™ COVID-19 RT-PCR assays immediately or stored at −20° C. until further processing.

### Assessment of Saliva's pH Neutralization With Dilution Buffer

The neutralization of saliva's pH was assessed with sodium hydroxide solution at different concentrations. Briefly, negative saliva samples presenting low or neutral pH were neutralized in different concentrations of sodium hydroxide (750 μM, 1 mM, and 1.5 mM). 1M NaOH stock solution was prepared by dissolving NaOH pellets in nuclease-free water. To test the detection sensitivity of SARS-CoV-2 in these dilutions, these saliva samples were spiked with 20 copies/μl of synthetic SARS-CoV-2 RNA (Twist Bioscience, San Francisco, CA), in a total of 50 copies per final reaction. The diluted saliva samples were then subjected to the RT-LAMP reaction, described below. After 30 minutes, the reactions were terminated and transferred to an ice-cold metal block for 30 seconds before the final color was read and a photograph was taken with a cell phone camera.

### SaliVISION™ Multiplex Primer Design

SARS-CoV-2 genomes spanning clades G, GH, GR, GV, L, O, S, V from GISAID were randomly collected and aligned with Mega7. In the predominantly expressed N gene, two non-overlapping conservative regions, namely N.1 and N.2, were selected as the testing targets. N.1 primers were designed with NEB LAMP Primer Design Tool, and a primer set located in N.2 region from a previous study was used to do a multiplex availability test with the N.1 primers with the ThermoFisher Multiple Primer Analyzer. A primer set for the wRNaseP gene from a previous study was used as the internal control. A 4 nucleotide Ploy A insert was introduced as a linker into all forward and back ward inner primers, in order to cooperate with the UDG treatment reaction to decrease the risk of contamination from previous reverse transcripts. BLASTN Somewhat Similar Alignment method was used to align the primers against the SARS-CoV-2 sequences from GISAID, to make sure there were no multiple mismatches for most of the genomes.

### SaliVISION™ Assay With the Colorimetric Reverse Transcription Loop-Mediated Isothermal Amplification (RT-LAMP)

Inactivated saliva samples were subjected to one-step reverse transcription loop-mediated isothermal amplification (RT-LAMP), using the customized multiplex primer set described herein that specifically targets two distinct regions on the N gene of SAR-CoV-2 viral genome. The inactivated and PH neutralized saliva samples were processed for RT-LAMP reactions using WarmStart® Colorimetric LAMP 2× Master Mix with UDG (New England Biolabs, Ipswich, MA), according to the manufacturer's protocol. Briefly, the assays were assembled in total reaction volumes of 40 μl, including 2.5 μl of original saliva sample, 20 μl of Warmstart® Colorimetric LAMP 2× Master Mix with UDG, 4 μl of 10× customized multiplex primer set, 1 μl of 1M guadinine hydrochloride, and filled up with RNA and DNA-free water. The reactions were incubated in the Fisherbrand™ Isotemp™ Digital Dry Block Heater (Fisher Scientific, Waltham, MA) set at 65° C. After 30 minutes, the reactions were terminated and transferred to an ice-cold metal block for 30 seconds before the final color was read and a photograph was taken with a cell phone camera.

### SalivaDirect™ RT-qPCR

For SARS-CoV-2 detection in saliva samples with the FDA EUA approved SalivaDirect™ assay, saliva specimens were aliquoted into 96-well plates, in the amount of 50 μl, and subsequently treated with 2.5 μl of 50 mg/mL proteinase K (AmericanBio, Canton, MA). Sample plates were shaken for 1 minute, wherein they were then incubated at 95° C. for 5 minutes for proteinase K inactivation. After that, the RT-qPCR was performed using TaqPath™ 1-Step RT-qPCR Master Mix (ThermoFisher Scientific, Waltham, MA) with SalivaDirect™ primer and probe set in a total volume of 20 μl per reaction, of which 5 μl was for the saliva sample. The samples were run using a Bio-Rad CFX96 Touch qPCR cycler. SalivaDirect™'s RT-PCR was used for viral nucleic acid detection with two probes: FAM probes for the presence of N1-gene amplicons, and Cy5 probes for the RNase P housekeeping gene. The threshold for positive samples was set at a Ct value of ≤40; however, exceptions were made based upon other result characteristics.

### ThermoFisher TaqPath™ COVID-19 RT-qPCR

200 μl of nasopharyngeal swab specimens collected in viral transport medium were aliquoted into a deep well plate, treated with a premixed solution containing of 265 μl of binding solution, 10 μl of total nucleic acid magnetic beads, 5 μl of 50 mg/mL proteinase K, and 5 μl of MS2 phage internal control (Applied BioSystems™, Foster City, CA), and then processed to RNA extraction with the KingFisher Flex automated extraction instrument (ThermoFisher Scientific, Waltham, CA), in accordance with manufacturer guidelines. 5 μl of RNA extract diluted in 40 μl of dilution buffer were subjected to qPCR using the TaqPath™ COVID-19 Combo Kit (ThermoFisher Scientific, Waltham, CA). The reactions were run with the Applied Biosystems 7500 Fast Dx Real-Time PCR System (ThermoFisher Scientific, Waltham, CA) to detect three viral probes (N-gene, S-gene, and the ORF1-gene) with the MS2 phage probe serving as an internal control. The threshold for positive samples was kept at a Ct value of ≤40, notwithstanding samples with Ct values of ≥40, present with extenuating circumstances.

### Statistical Analysis

All data were analyzed and graphed with GraphPad Prism 9 software (San Diego, CA). Specificity of the SaliVISION™ test was calculated as a percentage of the negative samples detected by RT-LAMP that were also negative in either SalivaDirect™ or TaqPath™ RT-PCR test. Sensitivity of a given Ct interval was calculated as the percentage of the positive samples detected by RT-LAMP hat were also positive in either SalivaDirect™ or TaqPath™ RT-PCR test. In both cases, 95% confidence intervals were calculated by interpreting the proportion of counts as binomial rates and then computed by the modified Wald method using GraphPad Prism 9 software.

### Protocol

### Saliva Collection

### Passive Drool:

1. Allow saliva to pool in mouth.
2. Open the cap and with head tilted forward, gently guide saliva through the provided collection tube pre-filled with 100 μl of Lysis Buffer.
3. Fill to the marked line (approximately 0.5 mL of saliva).
4. Securely close the cap and hand it to the clinical staff.

### Oral Swab:

1. Pull contents including Collector, Compression Cube, and Collection Tube pre-filled with 50 μl of Lysis Buffer on a clean and dry surface.
2. Attach the Collection Tube to the base of the Compression Tube and set aside.
3. Allow saliva to pool in mouth.
4 Place the tip of the pad of the Collector where saliva has pooled for 30 sec (slowly count from 1 to 30). The Collector may be removed from the mouth periodically to check the absorbance status, but resume collection immediately afterward.
5. Place the white absorbent pad end into the Compressor Tube, holding the Collector in an upright and vertical position, and slowly push the plunger downwards to transfer saliva from the pad into the Collection Tube until the saliva reaches the marked line on the Collection Tube.
6. Gently remove the Collection Tube and close the cap tightly.
7. Hand the Collection Tube to the clinical staff and dispose the Compression Tube containing the Collector in a proper biohazard waste container.

### Sample Processing

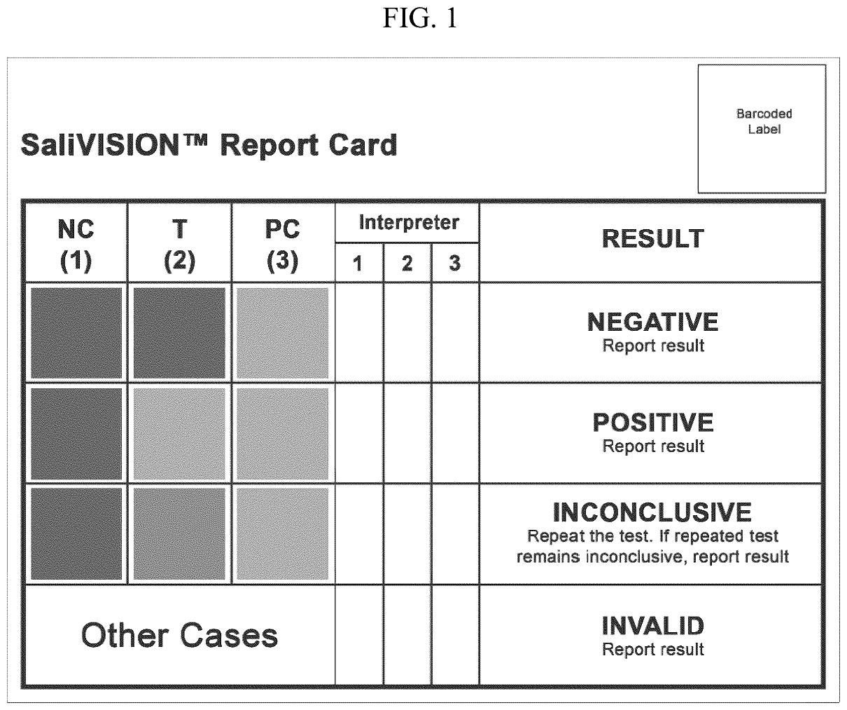

1. Vortex the Collection Tube briefly and incubate on a shaking heat block at 95° C. for 5 min at 1000 rpm.
2. Thaw the reaction mix at room temperature while it is still vacuumed sealed in the storage bag.
3. Cool the treated sample on ice for a few seconds.
4. Add 30 μl of treated saliva sample to #0 (DB, Dilution Buffer) and mix well.
5. Transfer 30 μl from #1 (NC, Negative Control) and mix well.
6. Transfer 30 μl from #1 to #2 (T, Test) and #3 (PC, Positive Control), without mixing.
7. Close the Tubes completely and invert a few times to mix the sample into the reaction mix.
8. Briefly centrifuge the tube for 5 sec and incubate on a heat block at 65° C. for 30 min.
9. Cool down the plate on ice for at least 30 sec and interpret the result using guideline in the Report Card (FIG. 1).

### Important Notes:

1). FRESHLY prepare the Multiplex Primer Sets (Table 3) right before preparing the reaction mixes.
2). ONLY prepare the reaction mixes within 48 hours in advance and store −20° C. (Table 4).
3). Vortex all reagents before drawing/adding to the mix.
4). The reaction mixes can be stored on dry ice ONLY in vacuum sealed bag for on-site use.
5). MINIMIZE the exposure of the LAMP Master Mix-containing reagents/reactions to the air.
6). When not in the vacuum sealed bag, the reaction mix should be stored on wet ice or cool block.
7). DO NOT open the reaction mixes after the amplification.
8). Use pipet tips with filter.
9). Take appropriate caution with all possible PCR contaminations.
10). Perform quality control (QC) with every new batch of reaction mix. The QC should include:
   No-template control
   No-primer control
   Negative saliva sample
   Negative saliva sample spiked with 50 copies of genomic SARS-CoV-2 RNA.

### Result Interpretation

Result interpretation will be performed independently by two trained staff. If the interpretation is not consistent, the 3rd interpreter will be required.
1. Examine the test's validity:
   a. The color of NC (#1) is PINK/RED and PC (#3) is YELLOW→VALID, proceed to step 2.
   b. Other cases→INVALID→report as INVALID.
2. If the test is valid, examine the color of T (#2):
   a. The color is PINK/RED→report as NEGATIVE
   b. The color is YELLOW→report as POSITIVE
   c. The color is ORANGE→INCONCLUSIVE→proceed to step 3.
3. If the test is inconclusive, repeat the test and extend the incubation time at 65° C. to 45 min.
   a. If the new result is as described in step 2a or b→report NEGATIVE or POSITIVE, accordingly.
   b. If the new result remains inconclusive→report as INCONCLUSIVE

### Selected Results

### Overview

Establishing a reliable means of readily available, rapid diagnostic testing for COVID-19 is of paramount importance in halting its spread. The current standard for laboratory testing utilizes reverse transcription quantitative polymerase chain reaction (RT-qPCR); however, this method presents clear limitations in requiring a longer run-time as well as reduced on-site testing capability. Therefore, we investigated the viability of an isothermal RT-multiplex amplification model for rapid COVID-19 diagnostic testing which is compatible with less invasive sample collection. Herein, a novel, two-step, reverse transcription loop-mediated isothermal amplification (RT-LAMP) assay, SaliVISION™, is reported.

Loop-mediated isothermal amplification (LAMP) is method in which nucleic acids (NAs) can be amplified under isothermal conditions, with increased specificity, and reaction times of less than 1 hour. Modification of the LAMP assay for RT-LAMP has offered a newmeans by which viral-genome amplification/detection can be utilized for rapid diagnostic testing of COVID-19. RT-LAMP offers a simplified amplification assay that is imbedded with a reporter system that allows for clear interpretation of results. Moreover, with specificity for the SARS-CoV-2 nucleocapsid (N) and spike (S) genes that is comparable to current lab testing methods, RT-LAMP has shown promise as a reliable method for viral detection of SARS-CoV-2. Therefore, the aforementioned benefits of RT-LAMP, coupled with its increased applicability in clinical point-of-care settings, suggest RT-LAMP as a viable option for large-scale rapid diagnostic testing of COVID-19.

The disclosed RT-LAMP assay employs a multiplex amplification approach, utilizing a 6-primer set (3 forward, 3 reverse) to create multiple reverse transcription start sites allowing for accelerated elongation. Specifically, the inner primers (FIP & BIP) bind to viral RNA and complementary DNA, respectively, whereby forward/reverse primers (F3 & B3) allow for multiple-site elongation. Subsequently, complementary sequences at specific positions allow for the DNA product to adopt a hairpin structure whereby the polymerase binds two loop-primers (LF & LB) for amplification of target viral genes. This process requires an RNA-dependent DNA polymerase with strong strand-displacement ability, and tolerance for increased temperatures—typically incubation at 65° C. for 30-40 minutes—which eliminates the need for a thermocycler with fluorescence detection. Instead, detection of viral RNA can be approached by a multitude of different methods using RT-LAMP; one of which is to use a pH indicator (e.g., phenol red) in a low-buffer reaction.

The SaliVISION™ Screening and Diagnostic Assay is a colorimetric method detecting an existing pathogen's viral RNA with loop-mediated isothermal amplification (LAMP) technology. The assay uses a phenol red pH reporter system wherein COVID-19-positive saliva samples induce color change following a 30-minute incubation period. During the amplification the production of protons, as a result of extensive DNA polymerase activity, subsequently causes a drop in pH, producing a change in solution color from pink to yellow, which can be visibly read out in 15-40 minutes, without a need of any specialized and expensive laboratory equipment. The test utilizes a customized multiplex primer set to detect multiple regions of a certain pathogen RNA. This two-step assay is designed for a rapid on-site performance directly on saliva samples, without a need of RNA extraction or laboratory equipment. The test result can be readily available by visualization within 40 minutes, based on the color change in the final reaction due to the change in pH after the amplification. The SaliVISION™ test has been tailored to meet specific requirements and urgent needs amid the COVID-19 pandemic, having a sensitivity of 2.5 copies/μl in the primary sample and a high specificity with no cross-reactivity to other human pathogens. Furthermore, the SaliVISION™ test can be performed in 40 minutes or less at any public location with minimal setup requirements, costs less than $12 a test, and is more comfortable than standard tests to detect COVID-19 since it uses saliva collection with a non-aggressive and simple process.

### Key Features of SaliVISION™

1) Primer design: Two overlapping regions of the N gene of the SARS-CoV-2 virus are used as the targets for the LAMP reaction primers, and there are 6 primers for each target. All 12 primers are used in a multiplex reaction tube to produce an intensified signal in order to increase the sensitivity. In the forward and backward inner primers, a polyA linker, composed of 4 adenine nucleotides is inserted in between the inner binding part and the reverse loop part of the primers. By using this design, the possible contamination causing amplicons will be eliminated more efficiently when they are exposed to the UDG enzyme because the broken loop will inhibit the initiation of artifact amplification.
2) Specificity: With the unique primer design as described above, the specificity of the test can be achieved with 100% confidence. In an example with a SARS-CoV-2 pathogen, the customized multiplex primer set had no cross-reactivity to common respiratory or other viral pathogens which would generate potential false positive results. This was confirmed by both an in silico analysis and a wet assay with the multiplex primer set against the N-gene of the SARS-CoV-2 virus.
3) Lysis buffer: The specialized lysis buffer used in the SaliVISION™ test is an all-in-one solution which serves two purposes: cell dissociation and RNA stabilization. The key components of this lysis buffer are Proteinase K (PK) and guanidine hydrochloride (GHCl). PK is a stable serine alkaline protease and has broad substrate specificity. GHCl is a strong denaturing agent which functions both to homogenize cells and denaturing endogenous RNases. The addition of GHCl significantly enhances the efficiency of the saliva's lysis process while protecting the RNAs from degradation during the test. The absence of GHCl in the lysis buffer will result in an inefficient cell dissociation (that requires longer time and/or higher concentration of PK) and a reduction in RNA integrity during the sample handling and processing steps.
4) Dilution strategy and reaction setup: Dilution Buffer with low concentration of NaOH is an important step to guarantee the success of the SaliVISION™ test. The normal range of human saliva has a pH of 6.2-7.4, with an average of 6.7. Additionally, approximately 10% of human saliva is acidic. This naturally low pH in the primary samples poses a significant problem in the development of a pH-dependent assay like SaliVISION™. To solve this problem, Dilution Buffer with a low concentration of NaOH must be used to 1) neutralize the natural acidity of the saliva sample and 2) stabilize the original pH of the reaction after sample input, without interfering with the sensitivity of the assay. In addition, a precise dilution factor is optimized to provide an adequate sample input to the final reaction, preventing inhibitory effect due to overwhelming concentration of sodium hydroxide and thus enhancing both efficiency and efficacy of the assay. The absence of this dilution step will cause pH instability in the reaction and thus increase the chance of having a false positive and/or invalid samples. In this test, the assay is designed to tolerate a saliva sample with a pH≥4.9 while still assuring the stability and reliability for on-site performance.

### Assessment of pH Neutralization in Original Saliva Samples

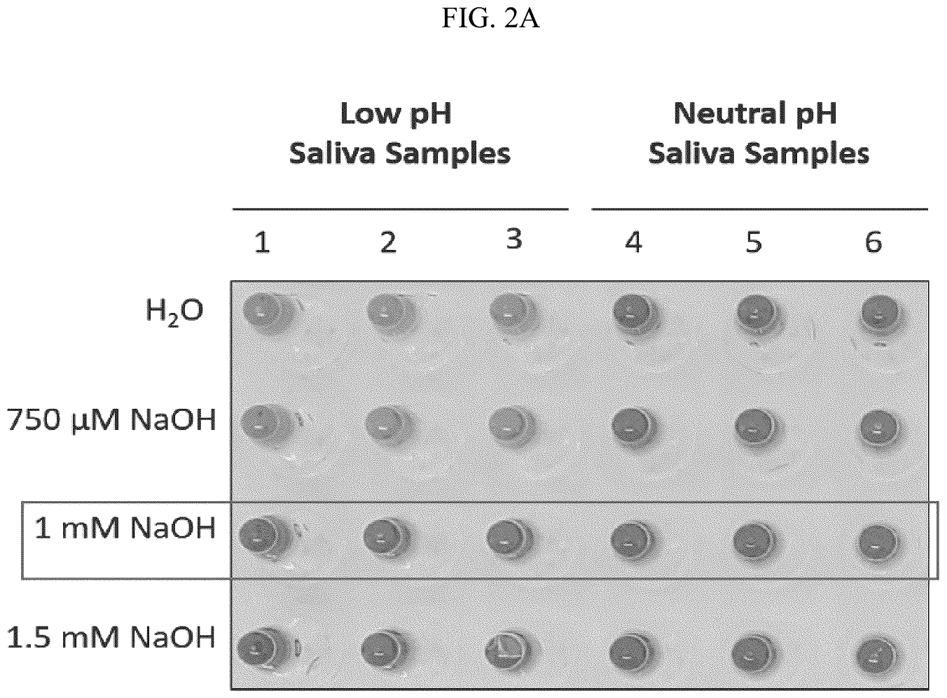

The normal range human saliva's pH is 6.2-7.4, with an average at 6.7. Additionally, approximately 10% of human saliva is acidic. This naturally low pH in primary samples pose a significant problem in the development of a pH-dependent assay, such as the SaliVISION™ test. To solve this problem, sodium hydroxide solution (NaOH) was tested for its ability to neutralize the acidity of human saliva. Saliva samples with low pH were diluted with different concentrations of NaOH solution. While the low pH saliva samples diluted in either H₂O or 750 μM NaOH caused a color change from pink to orange immediately after being added to the WarmStart® reaction mix, the color only showed a subtle change when these samples were diluted in 1 mM or 1.5 mM NaOH, indicating that higher concentration of NaOH could neutralize the acidity of saliva and thus maintain the original pH of the WarmStart® master mix prior the LAMP amplification (FIG. 2A). Strong base NaOH solution can neutralize the low pH in acidic saliva and enhance the specificity of the test. On the other hand, it may also result in increased alkalinity in saliva with neutral pH, and thus, reduce test sensitivity to these samples. To test this possibility, the SaliVISION™ assay was performed with spiked SARS-CoV-2 RNA in acidic and neutral saliva diluted with either 1 mM or 1.5 mM NaOH solution. At the NaOH concentration of 1 mM, all acidic and neutral saliva samples showed a strong positive result after 30 minutes of incubation at 65° C. The sensitivity of this assay, otherwise, was significantly diminished in neutral saliva samples diluted with 1.5 mM NaOH, when compared to the results of low pH saliva (FIG. 2B). The data showed that the Dilution Buffer with 1 mM concentration of NaOH is effective to neutralize the natural acidity of saliva sample and stabilize the original pH of the reaction after sample input, without interfering with the sensitivity of the assay. In this test, the assay is designed to tolerate saliva samples with pH≥4.9 while still ensuring stability and reliability for on-site performance.

### Limit of Detection (LoD)-Analytical Sensitivity

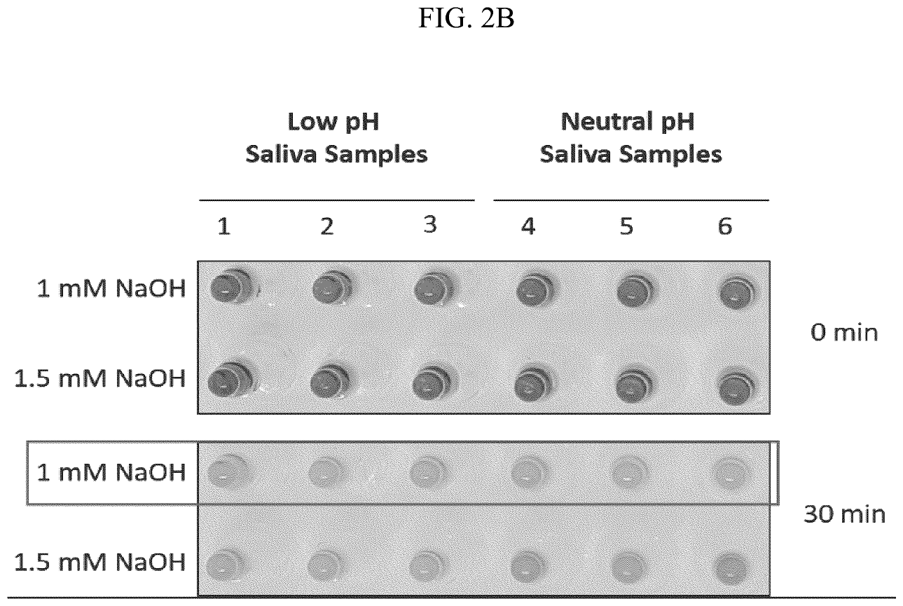

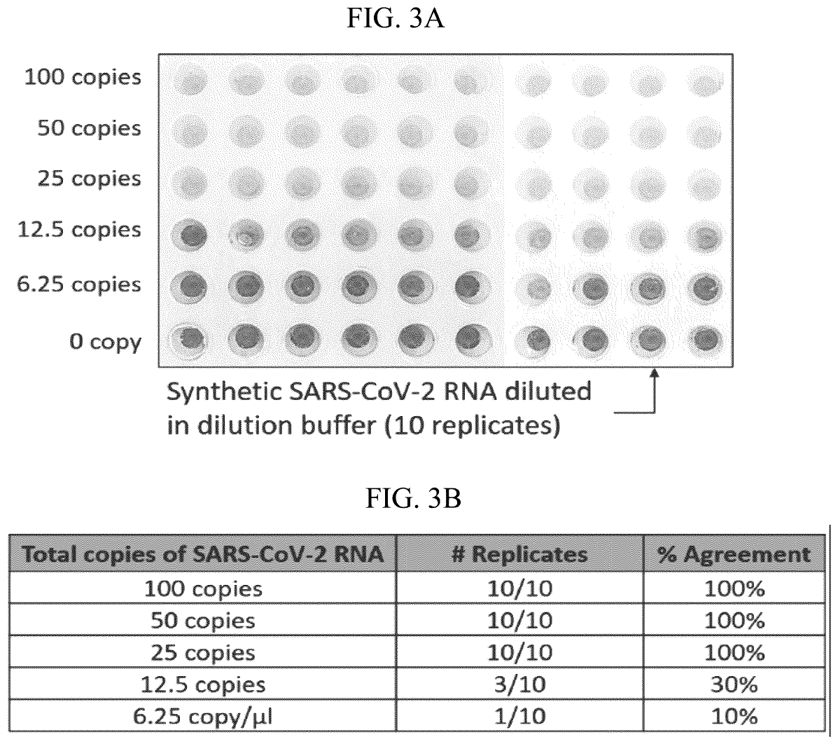

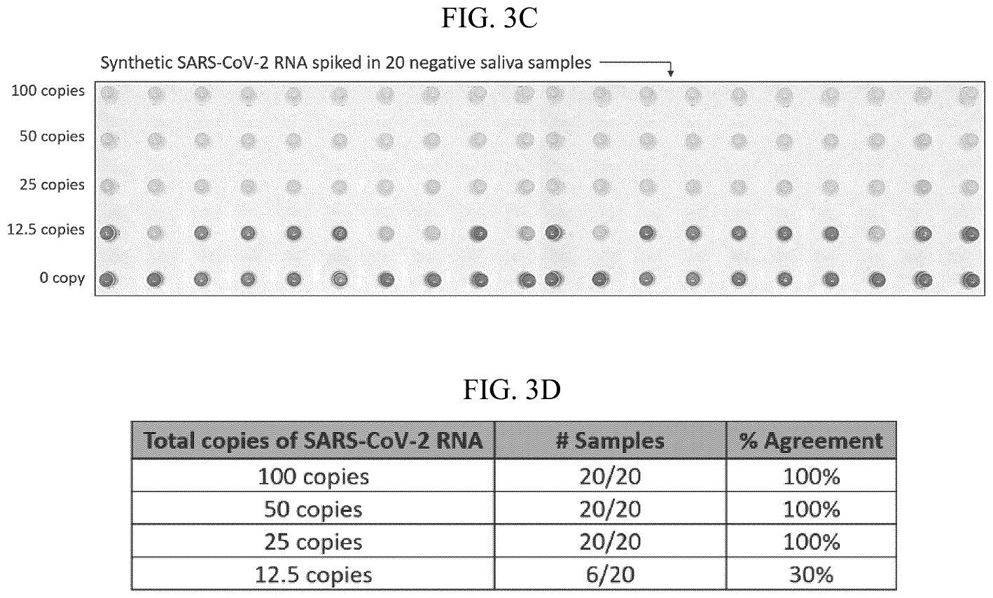

The assay for the LoD determination was performed with 5 points of 2-fold serial dilutions of the positive viral control (Transcripted SARS-CoV-2 genes provided by the ThermoFisher TagPath™ COVID-19 Combo Kit) spiked into Dilution Buffer. The final calculated viral concentrations for the contrived samples were 10 copies/μl, 5 copies/μl, 2.5 copies/μl, 1.25 copies/μl, and 0.625 copies/μl. The 2.5 copies/μl was determined as the lowest detectable concentration of genomic SAR-CoV-2 RNA, at which 100% of replicates were detected (FIGS. 3A-3B). An additional 10 samples containing only dilution buffer, served as negative controls and to rule out contamination (data not shown). Since 10 μl of sample input was used, the LoD allows for the detection of 25 copies of virus in the whole RT-LAMP reaction (Table 5).

TABLE 5
Limit of detection of SaliVISION ™

| | | |
|---|---|---|
| SARS-CoV-2 RNA | | |
| Concentration | Total Input | Sample Number |

| | | | | | |
|---|---|---|---|---|---|
| 10 | copies/μl | 100 | copies | 10/10 | (100%) |
| 5 | copies/μl | 50 | copies | 10/10 | (100%) |
| 2.5 | copies/μl | 25 | copies | 10/10 | (100%) |
| 1.25 | copies/μl | 12.5 | copies | 3/10 | (30%) |
| 0.625 | copies/μl | 6.25 | copies | 1/10 | (10%) |

The LoD of SaliVISION was subsequently verified on 20 negative saliva samples contrived with the same concentrations of synthetic SARS-CoV-2 RNA (Twist Bioscience, San Francisco, CA). These results were in 100% concordance with the results of the aforementioned analytical sensitivity assay, with 2.5 copies/μl being the lowest viral RNA concentration at which 100% of replicates were detected, therefore confirming the LoD of the SaliVISION assay (Table 6 and FIGS. 3C-3D).

TABLE 6
SaliVISION ™ LOD verification

| | | |
|---|---|---|
| SARS-CoV-2 RNA | | |
| Concentration | Total Input | Sample Number |

| | | | | | |
|---|---|---|---|---|---|
| 10 | copies/μl | 100 | copies | 20/20 | (100%) |
| 5 | copies/μl | 50 | copies | 20/20 | (100%) |
| 2.5 | copies/μl | 25 | copies | 20/20 | (100%) |
| 1.25 | copies/μl | 12.5 | copies | 6/20 | (30%) |

### Clinical Sensitivity

### Laboratory Performance:

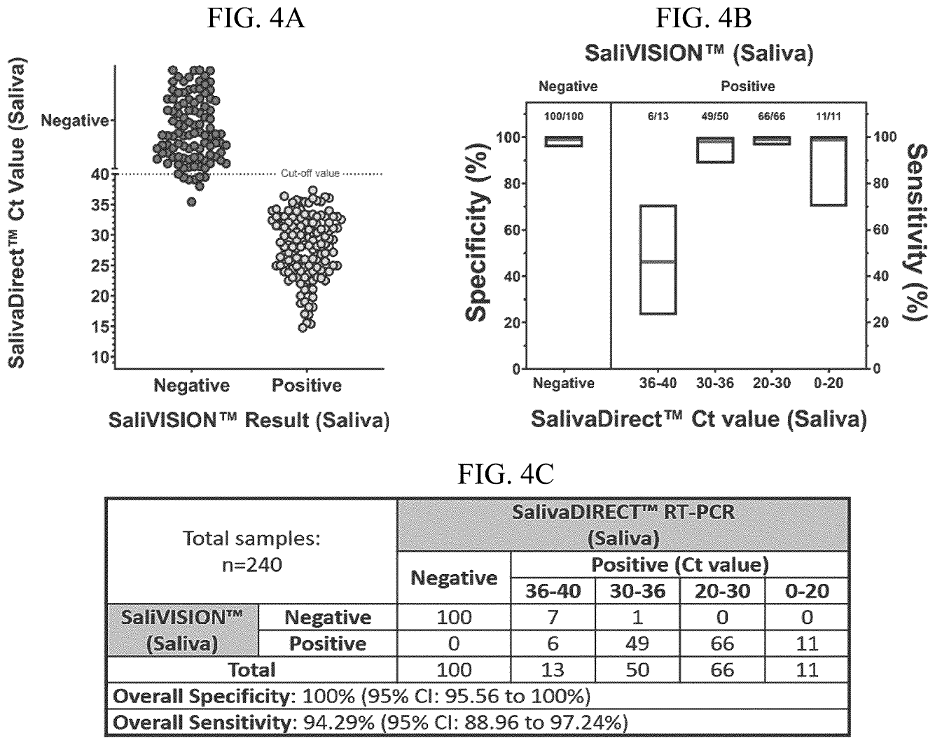

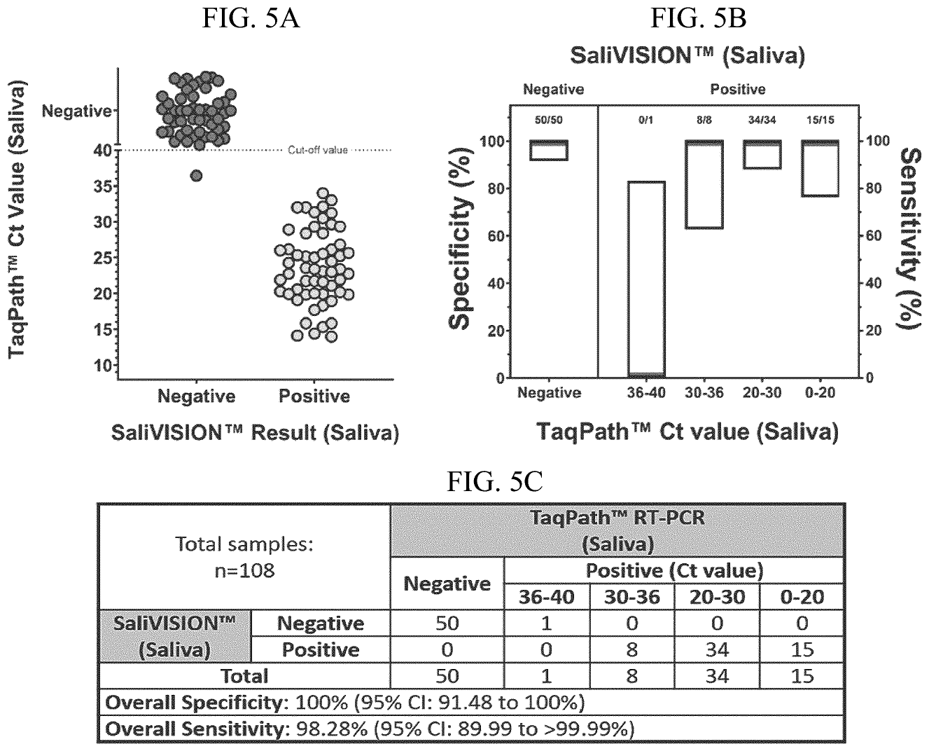

A clinical validation consisted of 240 clinical specimens that were clinically collected for SalivaDirect™ diagnostic test, including 140 positive specimens and 100 negative specimens. The results from the SaliVISION assay were blindly compared to the corresponding Ct values detected by the SalivaDirect™ test, wherein the Ct value refers to the cycle threshold (i.e. the number of cycles required for the fluorescent signal from the pH indicator to cross the threshold and exceed background level). All 100 saliva samples tested negative by SalivaDirect™ were negative by SaliVISION™ (100%). Out of 127 positive samples with Ct<36 detected by SalivaDirect™, 126 were positive by SaliVISION™ (99.21%). Out of 13 positive samples with Ct≥36, detected by SalivaDirect™, 6 of them were detected positive by SaliVISION™ (46.15%) (Table 7). Compared to SalivaDirect™ RT-PCR testing platform with saliva, the SaliVISION™ has an overall specificity of 100% (Wald's 95% CI: 95.56 to 100%) and an overall sensitivity of 94.29% (Wald's 95% CI: 88.96 to 97.24%) (FIGS. 4A-4C)

TABLE 7
Clinical validation of SaliVISION ™,
tested side-by-side with SalivaDirect ™
SaliVISION ™ Sensitivity in Comparison to SalivaDirect ™

| | Detected by | Detected by |
|---|---|---|
| Samples | SalivaDirect ™ | SaliVISION ™ |
| Positive (140) | Ct < 36: 127 | Ct < 36: 126/127 |
| | Ct ≥ 36: 13 | Ct ≥ 36: 6/13 |
| Negative (100) | 100 | 100/100 |

Negative Agreement: 100% (100/100)
Positive Agreement: 94.29% (132/140)

A clinical validation consisted of 108 saliva specimens previously collected for a COVID-19 diagnostic test, including 58 positive and 50 negative samples. The results from SaliVISION™ were compared to the corresponding Ct value detected by the Thermo Fisher TagPath™ RT-PCR COVID-19 test. All 50 saliva samples tested negative by TaqPath™ were negative by SaliVISION™ assay (100%). Out of 57 positive samples with Ct<36 detected by TaqPath™, all were positive by SaliVISION™ (100%). 1 positive sample with Ct≥36 detected by TaqPath™ was not detected by ≥(Table 8). Compared to TaqPath™ RT-PCR testing platform with saliva, the SaliVISION™ RT-LAMP test has an overall specificity of 100% (Wald's 95% CI: 91.48 to 100%) and an overall sensitivity of 98.28% (Wald's 95% CI: 89.99 to >99.99%) (FIGS. 5A-5C).

TABLE 8
Validation of SaliVISION ™ with Thermo Fisher
TaqPath ™ COVID-19 test (using saliva samples)
SaliVISION ™ Sensitivity in Comparison to TaqPath ™

| | Detected by | Detected by |
|---|---|---|
| Samples | TaqPath ™ | SaliVISION ™ |
| Positive (58) | Ct < 36: 57 | Ct < 36: 57/58 |
| | Ct ≥ 36: 1 | Ct ≥ 36: 0/1 |
| Negative (50) | 50 | 50/50 |

Negative Agreement: 100% (50/50)
Positive Agreement: 98.28% (57/58)

### Onsite Performance:

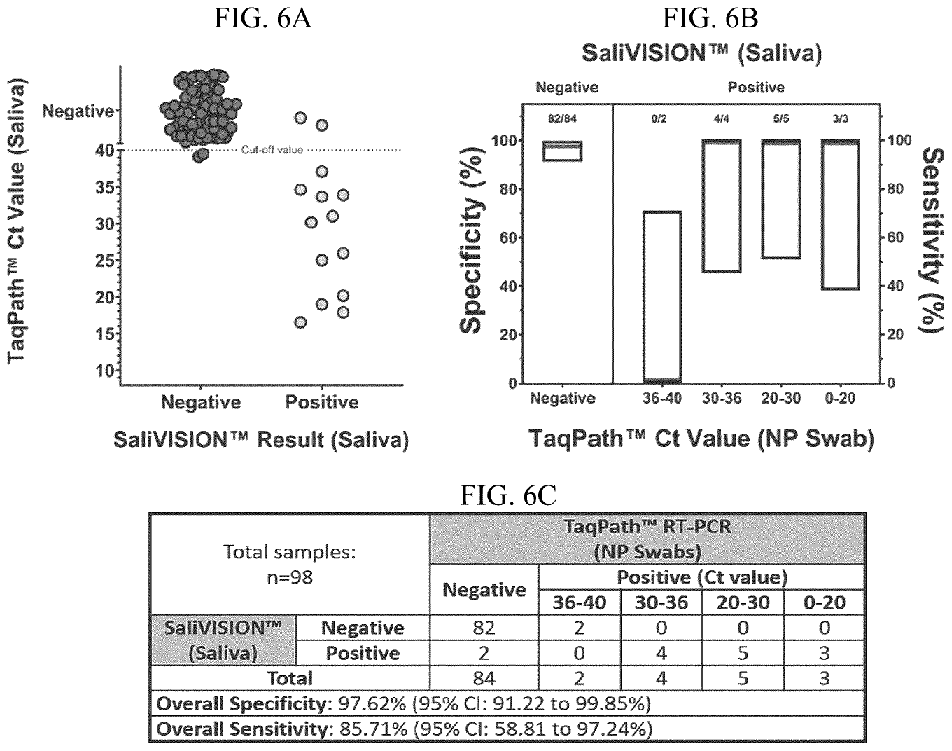

An on-site clinical validation was performed with saliva samples from 98 symptomatic individuals. The performance of SaliVISION™ was compared to the Thermo Fisher TaqPath™ RT-PCR COVID-19 platform by testing paired saliva and nasopharyngeal samples. Nasopharyngeal (NP) swabs and saliva were collected from symptomatic individuals. Saliva was collected either via passive drool in sterile 5 mL tubes, pre-loaded with Lysis Buffer, or Micro·SAL™ Saliva Collection Kit (Oasis Diagnostics Inc.). The SaliVISION™ assay was performed on-site and processed immediately after the collection. Verbal consent with a waiver of a signed consent form was provided to all patients for saliva collection. All 84 samples tested negative by the TaqPath™ COVID-19 test using NP swab specimens were negative by SaliVISION™ using saliva specimens (100%). Out of the 12 positive NP samples with Ct<36 detected by the Thermo Fisher TaqPath™ assay, all correlated saliva specimens were also positive by SaliVISION™ (100%). The 2 positive samples with Ct≥36 detected by the TaqPath™ assay were tested negative by the SaliVISION™ assay (Table 9). Compared to TaqPath RT-PCR testing plat form with corresponding NP swab samples, the SaliVISION™ RT-LAMP test has an overall specificity of 97.62% (Wald's 95% CI: 91.22 to 99.85%) and an overall sensitivity of 85.71% (Wald's 95% CI: 58.81 to 97.24%) (FIGS. 6A-6C). Although not wishing to be limited by theory, this inconsistency in detection may account for the difference in testing materials. In fact, several studies have shown that SARS-CoV-2 can be detected in the saliva of asymptomatic persons, while it was not detected in the corresponding NP swab samples. Of interest, a recent finding on Nature Medicine confirmed SARS-CoV-2 infection in the salivary glands and mucosae and thus, the oral cavity is an important site for SARS-CoV-2 infection.

TABLE 9
Clinical validation of SaliVISION ™, tested
side-by-side with Thermo Fisher TaqPath ™ COVID-19 test
(using saliva and nasopharyngeal swab samples, respectively)
SaliVISION ™ Sensitivity in Comparison to TaqPath ™ RT-PCR

| | Detected by | Detected by |
|---|---|---|
| Samples | TaqPath ™ | SaliVISION ™ |
| Positive (14) | Ct < 36: 12 | Ct < 36: 12/12 |
| | Ct ≥ 36: 2 | Ct ≥ 36: 0/2 |
| Negative (84) | 84 | 84/84 |

Negative Agreement: 100% (84/84)
Positive Agreement: 85.71% (12/14)

### Analytical Inclusivity Specificity

### Inclusivity In Silico Analysis of the SaliVISION™ Multiplex Primer Set:

Given the rapid evolution of pervasive, and in some cases, increasingly infectious SARS-CoV-2 variants, the robustness of COVID-19 testing methods is of paramount importance. To account for the potential presence of different SARS-CoV-2 strains, an in silico analysis was conducted for the inclusivity of the multiplex primer sets. In this multiplex LAMP reaction design, there are two primer sets targeting 2 non-overlapping N gene regions of SARS-CoV-2 sequence, set N1 and set N2, and there are 6 primers in each primer set. 200 complete high coverage SARS-CoV-2 sequences spanning clades G, GH, GR, GV, L, O, S, V from GISAID were randomly collected, with specimen collection time from December 2019 to November, 2020. BLASTN Somewhat Similar Alignment method was used to align the primers against the SARS-CoV-2 sequences.

BLASTN results showed that primer set N1 has a primer carrying 1 mismatch for 3% of the strains and another primer carrying 1 mismatch for 2% of the strains, while primer set N2 has a primer carrying 1 mismatch for 2% of the strains and another primer carrying 1 mismatch in 1% of the strains. None of the mismatches are at the 3′ end of the primers. No multiple mismatches were found simultaneously in the same primer set. The multiple mismatches causing false positivity is evaluated with low possibility (Table 10 and Table 11), therein revealing the SaliVISION™ primer sets to be of robust points of detection for the presence of SARS-CoV-2 viral RNA.

TABLE 10
In silico inclusivity analysis of Primer set N1

| Primer Type | FIP | BIP | F3 | B3 | LF | LB |
|---|---|---|---|---|---|---|
| Total # of SARS- | 200 | 200 | 200 | 200 | 200 | 200 |
| CoV-2 Strains | | | | | | |
| 100% Matched | | | 100% | 100% | 100% | 100% |
| 1 Mismatch | 3% | 2% | | | | |

TABLE 11
In silico inclusivity analysis of Primer set N2

| Primer Type | FIP | BIP | F3 | B3 | LF | LB |
|---|---|---|---|---|---|---|
| Total # of SARS- | 200 | 200 | 200 | 200 | 200 | 200 |
| CoV-2 Strains | | | | | | |
| 100% Matched | 100% | 100% | | 100% | | 100% |
| 1 Mismatch | | | 2% | | 1% | |

### Cross-Reactivity In Silico Analysis of SaliVISION Multiplex Primer Set:

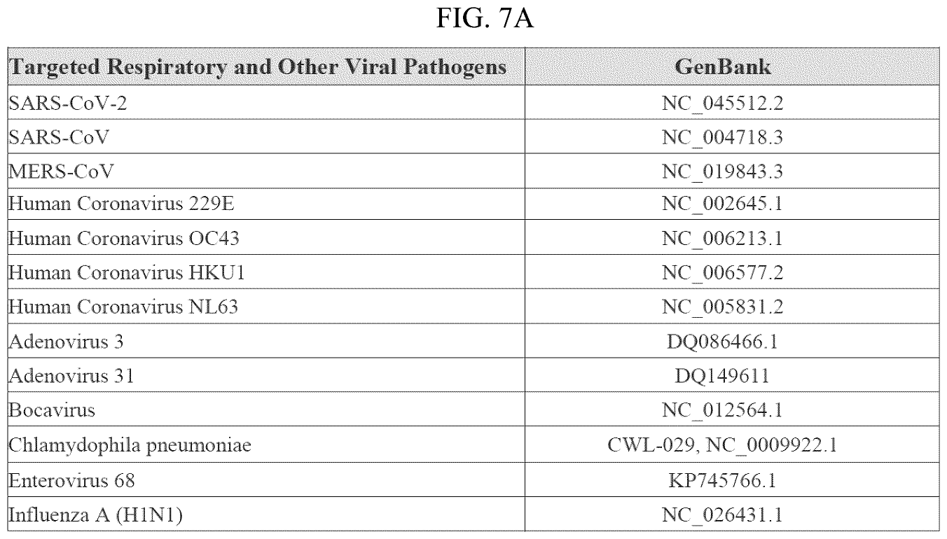

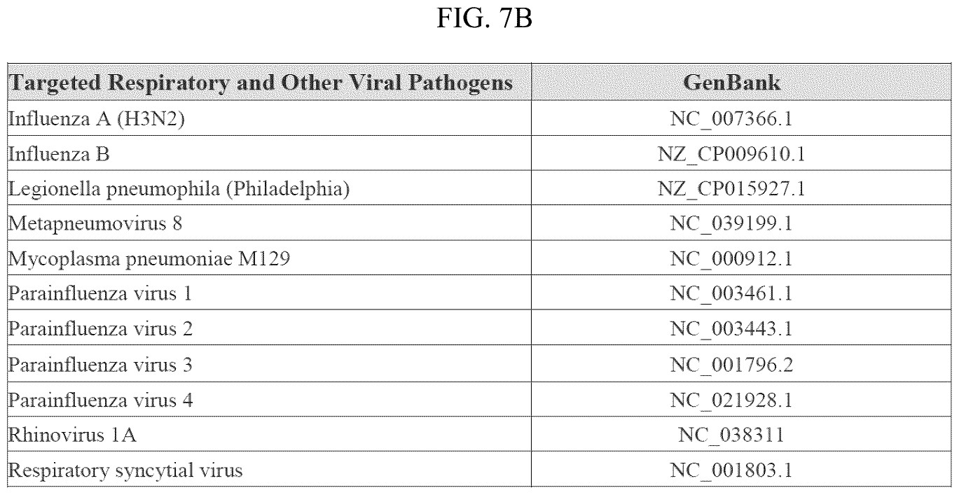

Due to the employment of large primer-sets in multiplex PCR assays, the success of a given protocol is largely contingent upon primer design. One such issue is cross-reactivity with other pathogens, given the potential overlap for any one of more than several pairs of primers. Therefore, to ensure the primer design provided adequate specificity for SARS-CoV-2, in silico cross-reactivity analyses of primer sequences was performed. In silico cross-reactivity analysis was performed by using BLASTN Somewhat Similar Alignment method to align the 12 primers against the 24 respiratory disease pathogens genomes, as listed in FIG. 2. The average nucleotide identity rate between 314 bps of primer sequences and SARS-CoV-2 is 100%. SARS-CoV has the average nucleotide identity rate 74.7% with the primer sequences, while in each primer set there is at least one primer has identity rate lower than 25% with SARS-CoV. All other pathogens do not have significant overall homologous sequences with the primers, except MERS-CoV has 66% identity rate with one single primer and Chlamydia pneumoniae CWL029 has 84% identity rate with another single primer. Since each primer set needs 6 primers to coordinate for a successful amplification, it is unlikely for these partial homologous primers to cause false positive (FIGS. 7A-7B).

### Cross-Reactivity Wet Testing of SaliVISION™ Multiplex Primer Set:

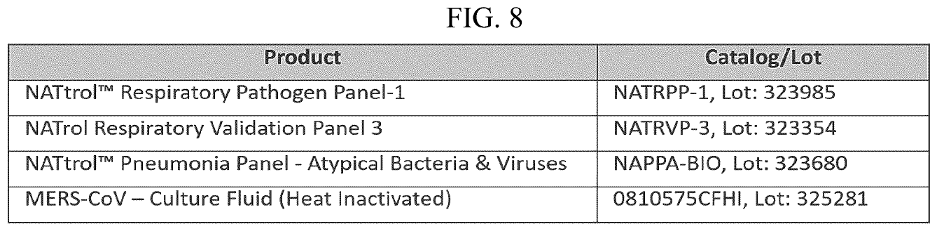

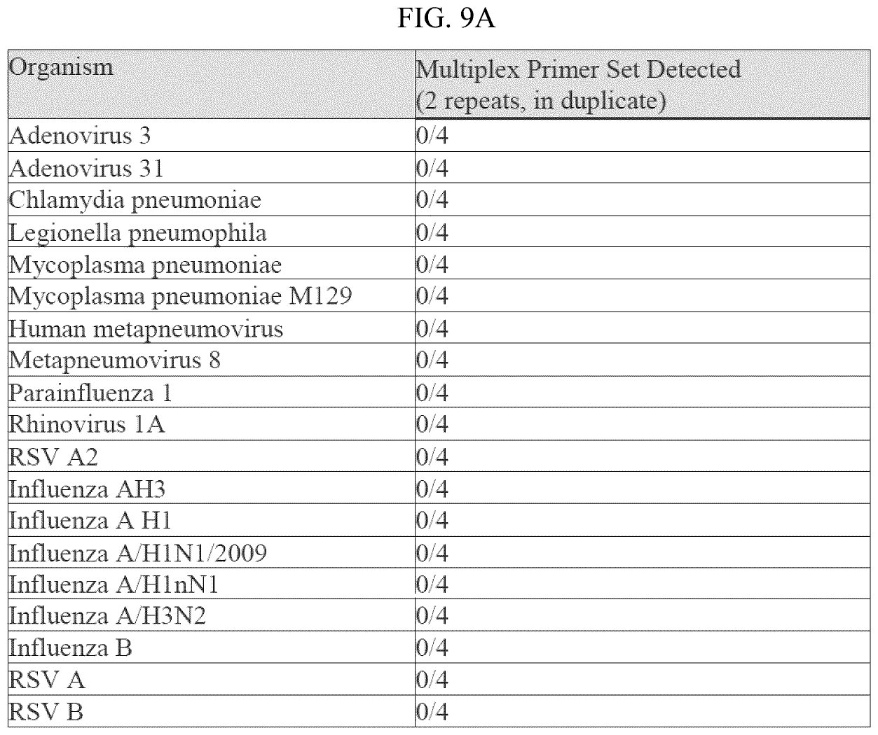

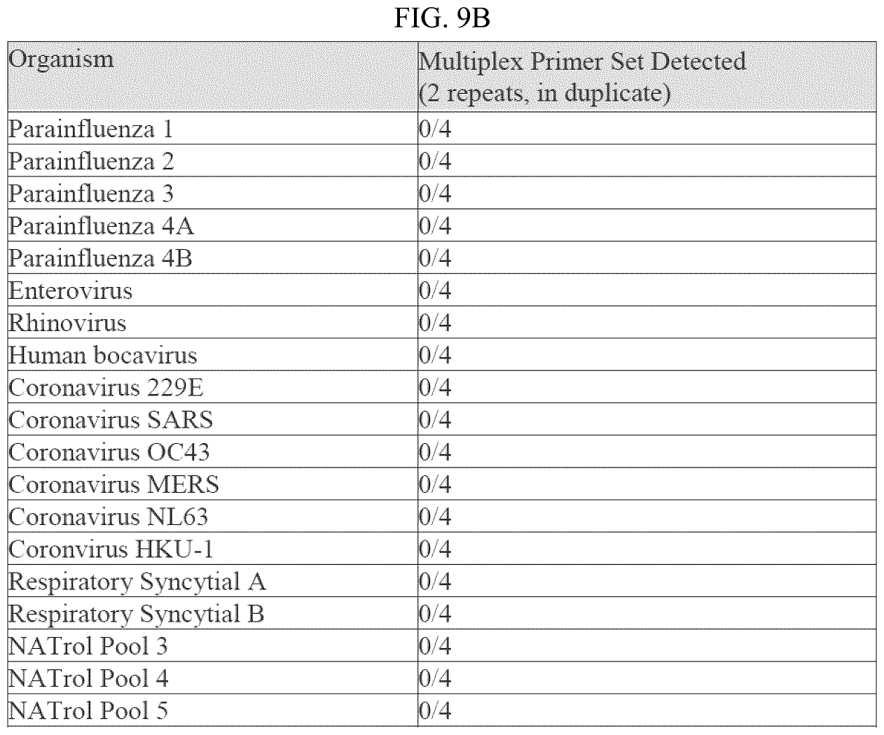

Following in silico analysis of the SaliVISION™ multiplex primer set, wet testing of the primer set was conducted to definitively rule out the possibility of cross-reactivity with the assay. The specificity of SaliVISION™ multiplex primer set was tested against common respiratory and other viral pathogens. This test was performed with purified, intact viral particles, cultured RNA, or bacterial cells commercially available at ZeptoMetrix Corporation (FIG. 8). 50 μl of each stock was spiked into a pooled negative saliva samples and tested with the SaliVISION™ assay. No positive signal was detected in any samples, indicating that this assay is specific for the detection of SARS-CoV-2, with no cross-reactivity to common respiratory or other viral pathogens which would generate potential false positive results (FIGS. 9A-9B).

### In Silico Analysis for the Inclusivity of SaliVISION™ Multiplex Primer Set:

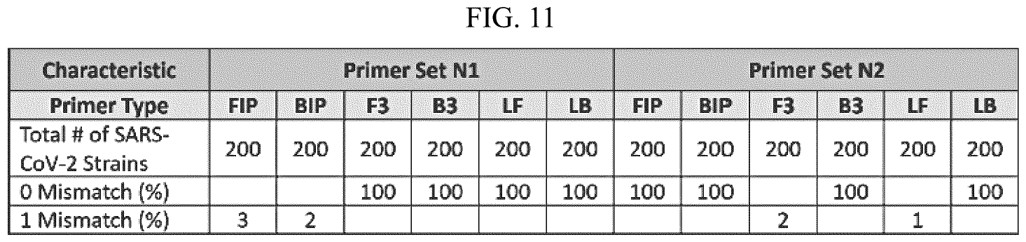

Given the rapid evolution of pervasive, and in some cases, increasingly infectious SARS-CoV-2 variants, the robustness of Covid-19 testing methods is of paramount importance. To account for the potential presence of different SARS-CoV-2 strains, we performed in silico analysis for the inclusivity of our multiplex primer sets. In our assay design, there are two primer sets targeting 2 non-overlapping N gene regions of the SARS-CoV-2 sequence, set N1 and set N2, with each primer set comprised of 6 primers. A total of 200 complete high coverage SARS-CoV-2 sequences spanning clades G, GH, GR, GV, L, O, S, V from GISAID were randomly collected, with specimen collection time from December 2019 to November 2020. BLASTN Somewhat Similar Alignment method was used to align the primers against the SARS-CoV-2 sequences. BLASTN results showed that the NI primer set has a primer carrying 1 mismatch for 3% of the strains and another primer carrying 1 mismatch for 2% of the strains. In comparison, the N2 primer set has a primer carrying 1 mismatch for 2% of the strains and another primer carrying 1 mismatch in 1% of the strains. None of the mismatches are at the 3′ end of the primers, and no multiple mismatches were found simultaneously in the same primer set. The multiple mismatches causing false positivity are evaluated with a low possibility, therein revealing the SaliVISION primer sets to be of robust points of detection for the presence of SARS-CoV-2 viral RNA (FIG. 11).

### Selected Conclusions

While public health efforts to fight the spread of COVID-19 have continuously yielded promising results, continued diagnostic testing persists as a foremost requirement in the prevention, and early detection of viral infection. Herein, a new, non-invasive RT-LAMP-based assay is disclosed for the rapid detection of SARS-CoV-2 in saliva, encompassing novel components for enhanced test accuracy. Following comparative analyses of the SaliVISION™ assay, in conjunction with both SalivaDirect™ and ThermoFisher TaqPath™ FDA EUA-approved diagnostic testing platforms, the SaliVISION™ assay offers 94.29% and 98.28% accuracy, respectively. Moreover, 100% specificity was reported for SARS-CoV-2, with no presentable cross-reactivity with other pathogens. Additionally, careful primer design for the targeted N-gene of the SARS-CoV-2 genome, with the inclusion of novel spacer-loop elements, have yielded greater amplification fidelity, and have contributed to the robustness of the test, in spite of a myriad of pervasive SARS variants. Furthermore, the versatility and scalability for which this assay presents allows for increased point-of-care and high-throughput testing capabilities, with a robustness that allows for consistent detection of SARS-CoV-2.

Thus far, molecular diagnostics that have been designed for the diagnosis of COVID-19 have presented an array of challenges which have been further compounded by a myriad of different factors. The primary challenge in the disclosed pH-based assay is that the original pH of the saliva samples can significantly impact to the stability of the reporter system and thus affect the specificity of the result. The normal range human saliva's pH is 6.2-7.4, with an average at 6.7. Additionally, approximately 10% of human saliva is acidic. The pH in these samples could reduce the pH of the WarmStart® master mix and cause a color change in the final reaction, despite the absence or presence of the SARS-CoV-2 DNA, leading to a false positive result. To solve this problem, the inactivated saliva samples were diluted with a defined concentration of NaOH to neutralize the 1) natural acidity of saliva sample, and 2) stabilize the original pH of the reaction after sample input, without interfering with the sensitivity of the assy. In addition, a precise dilution factor was optimized to provide an adequate sample input to the final reaction, preventing inhibitory effects due to overwhelming concentrations, thus enhancing both efficiency and efficacy of the assay. In this test, the assay is designed to tolerate saliva sample with pH≥4.9 while still ensuring adequate stability and reliability for on-site performance. In the interest of preventing cross-contamination, Uracil-DNA Gly-cosylase (UDG) was incorporated into the assay as a means of preventing amplicon contamination from antecedent reactions. Moreover, each reaction strip is completely closed prior to amplification and remains so as visualization of results occurs simultaneously with amplification. Lastly, because the success of RT-LAMP reactions is largely contingent upon successful primer design, several amendments to the primer sets, over the course of test development, were made necessary to maximize test specificity. As it stands, the SaliVISION™ assay provides 100% specificity for SARS-CoV-2, utilizing a two-primer set for distal locations on the N-gene. The specificity for which this test presents is also, in part, attributed to a novel spacer-loop element which is purposed for increased amplification fidelity for viral SARS-CoV-2 RNA.

Given the projected prevalence of asymptomatic persons carrying COVID-19, designated as asymptomatic spreaders, as well as the increase of new variants with more contagious characteristics, high testing sensitivity in molecular diagnostics for COVID-19 is of paramount importance. Moreover, as some studies have suggested high false-negative rates among RT-PCR diagnostics for COVID-19, the importance of high-sensitivity testing is further underscored as the continuation of false-negative diagnoses perpetuate the spread of COVID-19. RT-LAMP has since served as a popular alternative to traditional laboratory diagnostics due to its relative ease-of-use and overall versatility. However, current RT-LAMP tests typically provide a 75%-91% clinical sensitivity and offer a limit of detection—in congruence with the reported sensitivity—corresponding to a range of Ct values from ˜30-33.5. The SaliVISION™ assay has a clinical sensitivity, as confirmed on both SalivaDirect™ and TaqPath™ testing platforms, of 99.46% for saliva specimens with a Ct value of ≤36. While the standard convention for ruling out the possibility of COVID-19 infection is viral load in the amount corresponding to a Ct value of >36, the relative increase in sensitivity for which the SaliVISION™ assay provides, coupled with its ease-of-use, make it a reliable alternative to traditional laboratory diagnostics, which may be less cost and time efficient.

Saliva has become a promising alternative due to less invasive extraction procedures. Further, it has been reported that while saliva typically carries a lighter viral load, the SARS-CoV-2 virus is capable of infecting, and replicating, within cells lining the oral mucosa. In addition to infected cells of the lining of oral mucosa, it has also been observed that even in asymptomatic individuals, an acellular fraction of SARS-CoV-2 from infected glands is capable of making de novo virus, therein contributing to infectiousness as well as providing a source of live virus for saliva-based COVID-19 testing. Additionally, the viral longevity in saliva has been reported to be, on average, 18-20 days, which allows for saliva-based testing to be a valid diagnostic modality for the entire duration of an infected individual's contagious window. Therefore, saliva has become an appealing source for molecular diagnostic testing. When coupled with the associated non-invasive extraction procedures, as well as array of saliva-based rapid testing that currently exists, the use of saliva in COVID-19 testing is rapidly becoming an attractive alternative to other forms of testing.

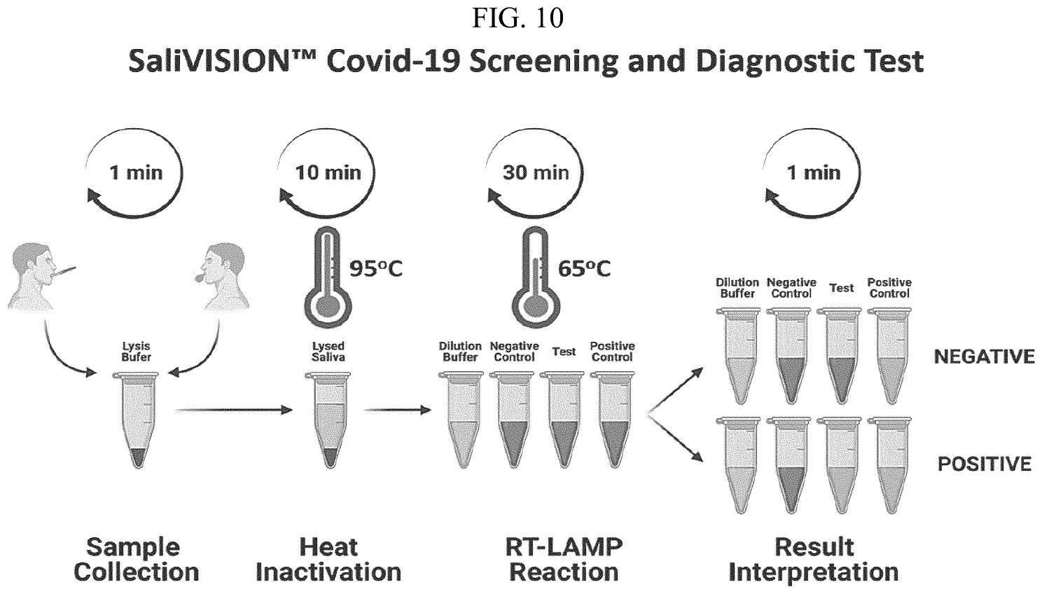

In the interest of expanding the scope of COVID-19 rapid saliva-based testing, it is imperative to establish a feasible methodology that encompasses stringent safety measures to prevent further spread of the virus. Currently, the body of rapid molecular diagnostics for COVID-19 is primarily comprised of antibody testing; however, such testing is markedly inadequate as the production COVID-19 specific antibodies may take up to several weeks, and thus will not inform an individual of active infection. Other rapid testing modalities include nucleic acid-based testing and antigen testing; the latter has yet to prove reliable in individuals present with low viral loads, while the former, albeit reliable, may still take up to a couple days for results, and is considerably more expensive. Given the foregoing, testing efforts, on all scales, would greatly benefit from an expanded repertoire of readily available rapid testing. The disclosed SaliVISION™ assay provides enhanced specificity and sensitivity when compared to other modes of rapid diagnostic testing, while proving more cost and time efficient, providing test results within 45 minutes. Moreover, an intuitive, convenient self-sample collection process is utilized that allows for increased throughput, while mitigating health-exposure risks (FIG. 10). Further, self-collection accommodation with the pre-loading of with lysis buffer inside the collection tube allows for specimens to be lysed and inactivated in a closed tube after the collection, thus circumventing the need for expensive biological safety cabinets and prolonged safety procedures. In congruence with these benefits, RT-LAMP provides a cheaper alternative testing method due to its minimalistic approach with regards to equipment and reagents. Therefore, the expansion of the RT-LAMP testing platform for the rapid diagnosis of COVID-19 is a promising avenue in the approach to ramping up large-scale testing.

As ongoing efforts to curb the spread of the COVID-19 global pandemic—such as mass vaccination—continue, the demand for readily accessible diagnostic testing remains to be of unequivocal importance, until the efficacy and accessibility of existing vaccines is better established. The early detection, and isolation of pre-symptomatic and asymptomatic individuals with COVID-19 is critical in mitigating the transmission of SARS-CoV-2. With its combined ease-of-use, accuracy, and rapid turn-around time, the SaliVISION™ assay has the potential to serve as a means of curbing the spread of the COVID-19 pandemic, particularly in stemming asymptomatic transmission. Given the versatility of the disclosed RT-LAMP assay, future work will entail modifying this technology to allow for the rapid diagnostic testing of other viral pathogens, for which detection can be made possible through salivary extracts.

### Enumerated Embodiments

The following enumerated embodiments are provided, the numbering of which is not to be construed as designating levels of importance.

Embodiment 1 provides a method of testing a subject for COVID-19, the method comprising:
obtaining a saliva sample from the subject;
diluting the saliva sample with a dilution buffer comprising a base;
contacting the diluted saliva sample with loop-mediated isothermal amplification (LAMP) primers that bind the N gene of the SARS-CoV-2 virus to form a test mixture under conditions that allow for reverse-transcription LAMP (RT-LAMP) of RNA material within the diluted saliva sample to take place;
   wherein the LAMP primer comprises an inner primer,
   wherein the reverse-transcribed RNA material comprises a first region which 5′ end is conjugated to the 3′ end of a second region which 5′ end is conjugated to the 3′ end of a DNA segment which is amplified by RT-LAMP;
   wherein the 3′ end of inner primer comprises a first sequence that is complementary to the first region, and the first sequence is conjugated through its 5′ end to the 3′ end of a second sequence that is identical to the second region;
   wherein a (A)ₙ polynucleotide, wherein n is 2, 3, 4, 5, 6, 7, 8, 9, or 10, is inserted between the first and second sequences within the inner primer; and
analyzing the color of the test mixture.

Embodiment 2 provides the method of embodiment 1, wherein the step of obtaining a saliva sample from the subject further comprises mixing the saliva sample with a lysis buffer.

Embodiment 3 provides the method of embodiment 2, wherein the lysis buffer comprises at least one of a serine alkaline protease and a denaturing agent.

Embodiment 4 provides the method of embodiment 3, wherein the serine alkaline protease is proteinase K and wherein the denaturing agent is guanidine hydrochloride.

Embodiment 5 provides the method of any one of embodiments 1-4, wherein the base is any alkaline or earth-alkaline hydroxide, such as but not limited to lithium hydroxide, sodium hydroxide, and/or potassium hydroxide, and/or ammonium hydroxide.

Embodiment 6 provides the method of any one of embodiments 1-5, wherein the LAMP primers comprise the following set of primers:

(FIP-N1; SEQ ID NO: 1)
TCCCCTACTGCTGCCTGGAGGAAAACAGTCAAGCCTCTTCTCG,
(BIP-N1, SEQ ID NO: 2)
CTCCTGCTAGAATGGCTGGCAAAAATCTGTCAAGCAGCAGCAAAG,
(F3-N1, SEQ ID NO: 3)
GCCAAAAGGCTTCTACGCA,
(B3-N1, SEQ ID NO: 4)
TTGCTCTCAAGCTGGTTCAA,
(LF-N1, SEQ ID NO: 5)
GCGACTACGTGATGAGGAA,
and
(LB-N1, SEQ ID NO: 6)
GGCGGTGATGCTGCTCTT.

Embodiment 7 provides the method of any one of embodiments 1-6, wherein the LAMP primers comprise the following set of primers:

(FIP-N2, SEQ ID NO: 7)
TGCGGCCAATGTTTGTAATCAGAAAACCAAGGAAATTTTGGGGAC,
(BIP-N2, SEQ ID NO: 8)
CGCATTGGCATGGAAGTCACAAAATTTGATGGCACCTGTGTAG,
(F3-N2, SEQ ID NO: 9)
AACACAAGCTTTCGGCAG,
(B3-N2, SEQ ID NO: 10)
GAAATTTGGATCTTTGTCATCC,
(LF-N2, SEQ ID NO: 11)
TTCCTTGTCTGATTAGTTC,
and
(LB-N2, SEQ ID NO: 12)
ACCTTCGGGAACGTGGTT.

Embodiment 8 provides the method of any one of embodiments 1-7, wherein the LAMP primers bind to two non-overlapping regions of the N gene of the SARS-CoV-2 virus.

Embodiment 9 provides the method of any one of embodiments 1-8, wherein the LAMP primers comprise each of SEQ ID NO:1 to SEQ ID NO:12.

Embodiment 10 provides the method of any one of embodiments 1-9, wherein the step of contacting the diluted saliva sample with one or more LAMP primers to form a test mixture further comprises centrifuging the test mixture and heating the test mixture at about 65° C. for about 30 minutes.

Embodiment 11 provides the method of any one of embodiments 1-10, wherein the step of analyzing the color of the test mixture comprises comparing the color of the test mixture to a positive control and a negative control,
wherein when the test mixture has the same color as the negative control, the subject does not have COVID-19,
when the test mixture has the same color as the positive control, the subject has COVID-19, and
when the test mixture has a color that is different than both the positive and negative controls, the result of the COVID-19 test is inconclusive.

Embodiment 12 provides the method of embodiment 11, wherein when the COVID-19 test is inconclusive,
the steps of obtaining a saliva sample from the subject, diluting the saliva sample with a dilution buffer comprising a base, and contacting the diluted saliva sample with one or more loop-mediated isothermal amplification (LAMP) primers that bind regions in the N gene of the SARS-CoV-2 virus to form a test mixture are repeated,
the step of contacting the diluted saliva sample with one or more LAMP primers to form a test mixture further comprises the steps of centrifuging the test mixture and heating the test mixture at about 65° C. for about 45 minutes, and
the step of analyzing the color of the test mixture is repeated.

Embodiment 13 provides a COVID-19 rapid-test kit comprising one or more components for the collection of a saliva sample from a subject, one or more components for processing of the saliva sample, and one or more components used to interpret the results of the COVID-19 test.

Embodiment 14 provides the COVID-19 rapid-test kit of embodiment 13, wherein the one or more components for the collection of a saliva sample comprise
a collection tube,
   lysis buffer comprising a serine alkaline protease, a denaturing agent, or a combination thereof, and
   an optional oral swab and compression tube.

Embodiment 15 provides the COVID-19 rapid-test kit of embodiment 14, wherein the serine alkaline protease is proteinase K and the denaturing agent is guanidine hydrochloride.

Embodiment 16 provides the COVID-19 rapid-test kit of any one of embodiments 13-15, wherein the one or more components for processing of the saliva sample comprise a dilution buffer comprising a base, primer sets corresponding to:

(a) the set:
(FIP-N1; SEQ ID NO: 1)
TCCCCTACTGCTGCCTGGAGGAAAACAGTCAAGCCTCTTCTCG,
(BIP-N1, SEQ ID NO: 2)
CTCCTGCTAGAATGGCTGGCAAAAATCTGTCAAGCAGCAGCAAAG,
(F3-N1, SEQ ID NO: 3)
GCCAAAAGGCTTCTACGCA,
(B3-N1, SEQ ID NO: 4)
TTGCTCTCAAGCTGGTTCAA,
(LF-N1, SEQ ID NO: 5)
GCGACTACGTGATGAGGAA,
and
(LB-N1, SEQ ID NO: 6)
GGCGGTGATGCTGCTCTT;
and/or
(b) the set:
(FIP-N2, SEQ ID NO: 7)
TGCGGCCAATGTTTGTAATCAGAAAACCAAGGAAATTTTGGGGAC,
(BIP-N2, SEQ ID NO: 8)
CGCATTGGCATGGAAGTCACAAAATTTGATGGCACCTGTGTAG,
(F3-N2, SEQ ID NO: 9)
AACACAAGCTTTCGGCAG,
(B3-N2, SEQ ID NO: 10)
GAAATTTGGATCTTTGTCATCC,
(LF-N2, SEQ ID NO: 11)
TTCCTTGTCTGATTAGTTC,
and
(LB-N2, SEQ ID NO: 12)
ACCTTCGGGAACGTGGTT.

Embodiment 17 provides the COVID-19 rapid-test kit of any one of embodiments 13-16, wherein the kit comprises primers of each of SEQ ID NO: 1 to SEQ ID NO: 12.

Embodiment 18 provides the COVID-19 rapid-test kit of any one of embodiments 13-17, wherein the one or more components for processing of the saliva sample further comprises one or more components for a positive and negative control,
wherein the one or more components for the negative control comprise DNA polymerase, reverse transcriptase, a pH indicator, dUTP, and Uracil-DNA Glycosylase (UDG), and
the one or more components for the positive control comprise DNA polymerase, reverse transcriptase, a pH indicator, dUTP, UDG, and RNase P gene primers selected from the group consisting of:

(FIP-R, SEQ ID NO: 13)
GTGTGACCCTGAAGACTCGGAAAAAGCCACTGACTCGGATC,
(BIP-R, SEQ ID NO: 14)
CCTCCGTGATATGGCTCTTCGAAAATTTCTTACATGGCTCTGGTC,
(F3-R, SEQ ID NO: 15)
TTGATGAGCTGGAGCCA,
(B3-R, SEQ ID NO: 16)
CACCCTCAATGCAGAGTC,
(LF-R, SEQ ID NO: 17)
ATGTGGATGGCTGAGTTGTT,
and
(LB-R, SEQ ID NO: 18)
CATGCTGAGTACTGGACCTC.

Embodiment 19 provides the COVID-19 rapid-test kit of any one of embodiments 13-18, wherein the negative control and the positive control both further comprise a portion of the saliva sample obtained from the subject in the dilution buffer.

Embodiment 20 provides the COVID-19 rapid-test kit of any one of embodiments 13-19, wherein the one or more components used to interpret the results of the COVID-19 test comprises a card showing the expected color of a positive, negative, and inconclusive COVID-19 test result.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of testing a subject for COVID-19, the method comprising:
obtaining a saliva sample from the subject;
diluting the saliva sample with a dilution buffer comprising a base;
contacting the diluted saliva sample with loop-mediated isothermal amplification (LAMP) primers that bind the N gene of the SARS-CoV-2 virus to form a test mixture under conditions that allow for reverse-transcription LAMP (RT-LAMP) of RNA material within the diluted saliva sample to take place;
wherein the LAMP primer comprises an inner primer,
wherein the reverse-transcribed RNA material comprises a first region which 5′ end is conjugated to the 3′ end of a second region which 5′ end is conjugated to the 3′ end of a DNA segment which is amplified by RT-LAMP;
wherein the 3′ end of inner primer comprises a first sequence that is complementary to the first region, and the first sequence is conjugated through its 5′ end to the 3′ end of a second sequence that is identical to the second region;
wherein a (A)ₙ polynucleotide, wherein n is 2, 3, 4, 5, 6, 7, 8, 9, or 10, is inserted between the first and second sequences within the inner primer; and
analyzing the color of the test mixture.

2. The method of claim 1, wherein the step of obtaining a saliva sample from the subject further comprises mixing the saliva sample with a lysis buffer.

3. The method of claim 2, wherein the lysis buffer comprises at least one of a serine alkaline protease and a denaturing agent.

4. The method of claim 3, wherein the serine alkaline protease is proteinase K and wherein the denaturing agent is guanidine hydrochloride.

5. The method of claim 1, wherein the base is sodium hydroxide.

6. The method of claim 1, wherein the LAMP primers comprise the following set of primers:
(FIP-N1; SEQ ID NO: 1)
TCCCCTACTGCTGCCTGGAGGAAAACAGTCAAGCCTCTTCTCG,
(BIP-N1, SEQ ID NO: 2)
CTCCTGCTAGAATGGCTGGCAAAAATCTGTCAAGCAGCAGCAAAG,
(F3-N1, SEQ ID NO: 3)
GCCAAAAGGCTTCTACGCA,
(B3-N1, SEQ ID NO: 4)
TTGCTCTCAAGCTGGTTCAA,
(LF-N1, SEQ ID NO: 5)
GCGACTACGTGATGAGGAA,
and
(LB-N1, SEQ ID NO: 6)
GGCGGTGATGCTGCTCTT.

7. The method of claim 1, wherein the LAMP primers comprise the following set of primers:
(FIP-N2, SEQ ID NO: 7)
TGCGGCCAATGTTTGTAATCAGAAAACCAAGGAAATTTTGGGGAC,
(BIP-N2, SEQ ID NO: 8)
CGCATTGGCATGGAAGTCACAAAATTTGATGGCACCTGTGTAG,
(F3-N2, SEQ ID NO: 9)
AACACAAGCTTTCGGCAG,
(B3-N2, SEQ ID NO: 10)
GAAATTTGGATCTTTGTCATCC,
(LF-N2, SEQ ID NO: 11)
TTCCTTGTCTGATTAGTTC,
and
(LB-N2, SEQ ID NO: 12)
ACCTTCGGGAACGTGGTT.

8. The method of claim 1, wherein the LAMP primers bind to two non-overlapping regions of the N gene of the SARS-CoV-2 virus.

9. The method of claim 8, wherein the LAMP primers comprise each of SEQ ID NO:1 to SEQ ID NO:12.

10. The method of claim 1, wherein the step of contacting the diluted saliva sample with one or more LAMP primers to form a test mixture further comprises centrifuging the test mixture and heating the test mixture at about 65° C. for about 30 minutes.

11. The method of claim 1, wherein the step of analyzing the color of the test mixture comprises comparing the color of the test mixture to a positive control and a negative control,
wherein when the test mixture has the same color as the negative control, the subject does not have COVID-19,
when the test mixture has the same color as the positive control, the subject has COVID-19, and
when the test mixture has a color that is different than both the positive and negative controls, the result of the COVID-19 test is inconclusive.

12. The method of claim 11, wherein when the COVID-19 test is inconclusive,
the steps of obtaining a saliva sample from the subject, diluting the saliva sample with a dilution buffer comprising a base, and contacting the diluted saliva sample with one or more loop-mediated isothermal amplification (LAMP) primers that bind regions in the N gene of the SARS-CoV-2 virus to form a test mixture are repeated,
the step of contacting the diluted saliva sample with one or more LAMP primers to form a test mixture further comprises the steps of centrifuging the test mixture and heating the test mixture at about 65° C. for about 45 minutes, and
the step of analyzing the color of the test mixture is repeated.

13. A COVID-19 rapid-test kit comprising one or more components for the collection of a saliva sample from a subject, one or more components for processing of the saliva sample, and one or more components used to interpret the results of the COVID-19 test.

14. The COVID-19 rapid-test kit of claim 13, wherein the one or more components for the collection of a saliva sample comprise
a collection tube,
lysis buffer comprising a serine alkaline protease, a denaturing agent, or a combination thereof, and
an optional oral swab and compression tube.

15. The COVID-19 rapid-test kit of claim 14, wherein the serine alkaline protease is proteinase K and the denaturing agent is guanidine hydrochloride.

16. The COVID-19 rapid-test kit of claim 13, wherein the one or more components for processing of the saliva sample comprise
a dilution buffer comprising a base,
primer sets corresponding to:
(a) the set:
(FIP-N1; SEQ ID NO: 1)
TCCCCTACTGCTGCCTGGAGGAAAACAGTCAAGCCTCTTCTCG,
(BIP-N1, SEQ ID NO: 2)
CTCCTGCTAGAATGGCTGGCAAAAATCTGTCAAGCAGCAGCAAAG,
(F3-N1, SEQ ID NO: 3)
GCCAAAAGGCTTCTACGCA,
(B3-N1, SEQ ID NO: 4)
TTGCTCTCAAGCTGGTTCAA,
(LF-N1, SEQ ID NO: 5)
GCGACTACGTGATGAGGAA,
and
(LB-N1, SEQ ID NO: 6)
GGCGGTGATGCTGCTCTT;
and/or
(b) the set:
(FIP-N2, SEQ ID NO: 7)
TGCGGCCAATGTTTGTAATCAGAAAACCAAGGAAATTTTGGGGAC,
(BIP-N2, SEQ ID NO: 8)
CGCATTGGCATGGAAGTCACAAAATTTGATGGCACCTGTGTAG,
(F3-N2, SEQ ID NO: 9)
AACACAAGCTTTCGGCAG,
(B3-N2, SEQ ID NO: 10)
GAAATTTGGATCTTTGTCATCC,
(LF-N2, SEQ ID NO: 11)
TTCCTTGTCTGATTAGTTC,
and
(LB-N2, SEQ ID NO: 12)
ACCTTCGGGAACGTGGTT.

17. The COVID-19 rapid-test kit of claim 16, wherein the kit comprises primers of each of SEQ ID NO:1 to SEQ ID NO: 12.

18. The COVID-19 rapid-test kit of claim 16, wherein the one or more components for processing of the saliva sample further comprises one or more components for a positive and negative control,
wherein the one or more components for the negative control comprise DNA polymerase, reverse transcriptase, a pH indicator, dUTP, and Uracil-DNA Glycosylase (UDG), and
the one or more components for the positive control comprise DNA polymerase, reverse transcriptase, a pH indicator, dUTP, UDG, and RNase P gene primers selected from the group consisting of:
(FIP-R, SEQ ID NO: 13)
GTGTGACCCTGAAGACTCGGAAAAAGCCACTGACTCGGATC,
(BIP-R, SEQ ID NO: 14)
CCTCCGTGATATGGCTCTTCGAAAATTTCTTACATGGCTCTGGTC,
(F3-R, SEQ ID NO: 15)
TTGATGAGCTGGAGCCA,
(B3-R, SEQ ID NO: 16)
CACCCTCAATGCAGAGTC,
(LF-R, SEQ ID NO: 17)
ATGTGGATGGCTGAGTTGTT,
and
(LB-R, SEQ ID NO: 18)
CATGCTGAGTACTGGACCTC.

19. The COVID-19 rapid-test kit of claim 18, wherein the negative control and the positive control both further comprise a portion of the saliva sample obtained from the subject in the dilution buffer.

20. The COVID-19 rapid-test kit of claim 13, wherein the one or more components used to interpret the results of the COVID-19 test comprises a card showing the expected color of a positive, negative, and inconclusive COVID-19 test result.


SEQUENCE LISTING (18 sequences)

SEQ ID NO: 1 - DNA, 43 bp, Artificial Sequence
  FIP-N1
tcccctactgctgcctggaggaaaacagtcaagcctcttctcg

SEQ ID NO: 2 - DNA, 45 bp, Artificial Sequence
  BIP-N1
ctcctgctagaatggctggcaaaaatctgtcaagcagcagcaaag

SEQ ID NO: 3 - DNA, 19 bp, Artificial Sequence
  F3-N1
gccaaaaggcttctacgca

SEQ ID NO: 4 - DNA, 20 bp, Artificial Sequence
  B3-N1
ttgctctcaagctggttcaa

SEQ ID NO: 5 - DNA, 19 bp, Artificial Sequence
  LF-N1
gcgactacgtgatgaggaa

SEQ ID NO: 6 - DNA, 18 bp, Artificial Sequence
  LB-N1
ggcggtgatgctgctctt

SEQ ID NO: 7 - DNA, 45 bp, Artificial Sequence
  FIP-N2
tgcggccaatgtttgtaatcagaaaaccaaggaaattttggggac

SEQ ID NO: 8 - DNA, 43 bp, Artificial Sequence
  BIP-N2
cgcattggcatggaagtcacaaaatttgatggcacctgtgtag

SEQ ID NO: 9 - DNA, 18 bp, Artificial Sequence
  F3-N2
aacacaagctttcggcag

SEQ ID NO: 10 - DNA, 22 bp, Artificial Sequence
  B3-N2
gaaatttggatctttgtcatcc

SEQ ID NO: 11 - DNA, 19 bp, Artificial Sequence
  LF-N2
ttccttgtctgattagttc

SEQ ID NO: 12 - DNA, 18 bp, Artificial Sequence
  LB-N2
accttcgggaacgtggtt

SEQ ID NO: 13 - DNA, 41 bp, Artificial Sequence
  FIP-R
gtgtgaccctgaagactcggaaaaagccactgactcggatc

SEQ ID NO: 14 - DNA, 45 bp, Artificial Sequence
  BIP-R
cctccgtgatatggctcttcgaaaatttcttacatggctctggtc

SEQ ID NO: 15 - DNA, 17 bp, Artificial Sequence
  F3-R
ttgatgagctggagcca

SEQ ID NO: 16 - DNA, 18 bp, Artificial Sequence
  B3-R
caccctcaatgcagagtc

SEQ ID NO: 17 - DNA, 20 bp, Artificial Sequence
  LF-R
atgtggatggctgagttgtt

SEQ ID NO: 18 - DNA, 20 bp, Artificial Sequence
  LB-R
catgctgagtactggacctc